(19) United States
(12) Patent Application Publication
(10) Pub. No.: US 2023/0055457 A1
(43) Pub. Date: Feb. 23, 2023

(54) CELL EXPANSION METHODS FOR PREVENTING CELL SENESCENCE AND PRESERVING THE THERAPEUTIC POTENCY OF HUMAN MESENCHYMAL STEM CELLS

(71) Applicants: Teng MA, Tallahassee, FL (US); Xuegang YUAN, Tallahassee, FL (US); Timothy LOGAN, Tallahassee, FL (US); THE FLORIDA STATE UNIVERSITY RESEARCH FOUNDATION, INC., Tallahassee, FL (US)

(72) Inventors: Teng MA, Tallahassee, FL (US); Xuegang YUAN, Tallahassee, FL (US); Timothy LOGAN, Tallahassee, FL (US)

(21) Appl. No.: 17/793,360

(22) PCT Filed: Jan. 15, 2021
(86) PCT No.: PCT/US21/13554

§ 371 Date: Jul. 15, 2022

Related U.S. Application Data
(60) Provisional application No. 62/961,281, filed on Jan. 15, 2020.

(51) Int. Cl. C12N 5/0775

(52) U.S. Cl. CPC C12N 5/0663; C12N 2500/98; C12N 2500/38

(57) ABSTRACT

Disclosed are compositions and methods for culturing cells in a manner that inhibits culture induced senescence. In one aspect, the methods comprise the administration of nicotinamide adenine dinucleotide (NAD) precursors to the culture. Further disclosed are NAD precursors used in the methods, comprising nicotinamide (NAM), nicotinamide mononucleotide (NMN), and/or nicotinamide riboside (NR).

This application claims the benefit of U.S. Provisional Application No. 62/961,281, filed on Jan. 15, 2020, which is incorporated herein by reference in its entirety.

This invention was made with government support under Grant No. CBET 1743426 awarded by the National Science Foundation. The Government has certain rights in the invention.

### I. BACKGROUND

Human mesenchymal stem cells (hMSCs) isolated from various adult tissues are primary candidates in cell therapy and being tested in clinical trials for a wide range of diseases. The pro-regenerative and therapeutic properties of hMSCs are largely attributed to their trophic effects that coordinately modulate the progression of inflammation and enhance the endogenous tissue repair by host progenitor cells. However, after isolation in vitro culture expansion, hMSCs lose their in vivo quiescent state and start to accumulate genetic and phenotypic changes that significantly alter their phenotypic properties with reduced therapeutic potential. Due to the Hayfilck limit, hMSCs went into cellular senescence after replicative culture expansion in vitro. Within the senescent state, the proliferation rate was significantly reduced and hMSCs can only maintain the basic cellular function and lose cellular homeostasis. During replicative senescence, DNA damage, cell cycle arrest, and dysfunction of cellular compartments would occur and further influence hMSC stemness and therapeutic potency. Since large-scale production of hMSCs is a basic requirement in clinical or industrial application, maintaining cellular function and homeostasis during replicative culture expansion of hMSCs becomes the major barrier for hMSC large-scale manufacturing. Thus, what is needed is culture methods and reagents that reduce culture induced senescence of hMSC and further preserve cellular homeostasis during culture expansion.

### II. SUMMARY

Disclosed are methods and compositions related to culturing in a manner that rescues, inhibits, prevents, or reduces culture induced senescence.

In one aspect, disclosed herein are methods of culturing and/or expanding cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts); preventing, inhibiting, and/or reducing culture induced senescence of a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts); and/or rescuing a cell or cell line such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts) from senescence during culture and expansion, said methods comprising placing cell or cell line in a media comprising a nicotinamide adenine dinucleotide (NAD+) precursor (such as, for example, nicotinamide (NAM), nicotinamide mononucleotide (NMN), and/or nicotinamide riboside (NR)). In one aspect, the NAD+ precursor can be added to the culture media after culture induced senescence has been detected. In some aspect, the glycolysis in the culture is enhanced.

Also disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the NAD precursor is added to the culture after passage (P) 1 (P1), P2, P3, P4, P5, P6, P7, P8, P9, P10, P11, P12, P13, P14, P15, P16, P17, P18, P19, P20, P21, P22, P23, P24, P25, P26, P27, P28, P29, P30, P35, P40, P45, P50, P55, P60, P65, P70, P75, P80, P85, P90, P95, or P100.

In one aspect, disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the NAD precursor is added to the culture at least one time every 12, 24, 36, 48, 60, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 60 days. For example, disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the NAD precursor is added to the culture at least 1, 2, 3, 4, 5, 6, 7, 8, 9, 10, 11, 12, 13, or 14 times per week. In some aspects, the NAD precursor is added to the culture at least 1, 2, 3, 4, 5, 6, 7, 8, 9, 10, 11, 12, 13, or 14 times per passage of culture.

Also disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the cells are passaged at least once every 12, 18, 24, 30, 36, 42, 48, 54, 60, 66, 72, 78, 84, 90, 96, 102, 108, 114, 120, 126, 132, 138, 144, 150, 156, 162, or 168 hours.

In one aspect, disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the media is a Xeno-free culture medium.

Also disclosed herein is culture media comprising one or more NAD precursors (such as, for example, nicotinamide (NAM), nicotinamide mononucleotide (NMN), and/or nicotinamide riboside (NR)).

In one aspect, disclosed herein is NAD precursor comprising culture media of any preceding aspect, wherein the media comprises Roswell Park Memorial Institute (RPMI) 1640, complete culture medium (CCM), Minimal Essential Medium (MEM), Dulbecco's Modified Eagle Medium (DMEM), Iscove's Modified Dulbecco's Medium (IMDM), Eagle's mimumal essential medium (EMEM), Cell Therapy systems (CTS) essential 8 medium, Medium 199, essential 8 medium, StemFlex medium, and AdvanceSTEM cell culture media. In some aspects, the media is a Xeno-free culture medium.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, 1H, 1I, 1J, and 1K show in vitro culture expansion of hMSCs results in cellular senescence breakdown of cellular homeostasis. FIG. 1A shows the alteration of hMSC morphology during culture expansion. FIG. 1B shows SA-beta-Gal activity and (1C) SA-beta-Gal staining in early passage and late passage of hMSCs. FIG. 1D shows that the comet assay demonstrated DNA damage of hMSCs during culture expansion. FIG. 1E shows population doubling (PD) time was prolonged for long-term cultured hMSCs. FIG. 1F shows that colony-forming unit (CFU-F) ability decreased during culture expansion of hMSCs. FIG. 1G shows mRNA levels of stem cell genes and (1H) mRNA levels of cell cycle gene expression in late passage of hMSCs compare to early passage. FIG. 1I shows cell cycle analysis of hMSCs via flow cytometry during culture expansion. FIG. 1J shows autophagy gene expression of hMSCs during culture expansion. FIG. 1K shows basal autophagic flux was reduced in late passage of hMSCs compare early passage. Late passage of hMSCs: passage 12-13, early passage of hMSCs: passage 4-5. Scale bar is 100 μm. *, p<0.05; **, p<0.01; ***, p<0.001.

FIGS. 2A, 2B, 2C, 2D, 2E, 2F, 2G, 2H, and 2I show culture expansion of hMSCs induced mitochondrial dysfunction. FIG. 2A shows mitochondrial morphology was altered during culture expansion of hMSCs. Culture expansion of hMSCs resulted in (2B) increased mitochondrial mass, and (2C) decreased mitochondrial membrane potential (MMP), determined by flow cytometry. FIG. 2D shows mitochondrial reactive oxygen species (ROS) and (2E) total ROS (determined by flow cytometry) were also increased during culture expansion of hMSCs. FIG. 2F shows electron transport chain complex I activity was also reduced in late passage of hMSCs. FIG. 2G shows loss of hMSC mitophagy ability induced by culture expansion. FIG. 2H shows RT-PCR analysis of genes involved in mitochondrial fusion and fission dynamics. FIG. 2I shows mRNA levels of genes involved in mitochondrial biogenesis during expansion of hMSCs. Scale bar is 50 μm. *, p<0.05; **, p<0.01; ***, p<0.001.

FIGS. 3A, 3B, 3C, 3D, 3E, 3F, 3G, 3H, 3I, 3J, 3K, 3L, 3M, and 3N show that culture expansion induces hMSC metabolic reconfiguration. FIG. 3A shows that glycolytic ATP ratio was changed during culture expansion of hMSCs. FIG. 3B shows gene expression of glycolysis and PPP pathways of hMSCs during culture expansion. FIG. 3C shows lactate production/glucose consumption ratio of hMSCs during replicative expansion. GC-MS analysis of hMSCs during culture expansion: FIG. 3D shows internal normalized peak area of lactate. (3E) absolute total molar percent enrichment (ATMPE) of ¹³C-glucose atoms in metabolites involved in glycolysis and OXPHOS. FIG. 3F shows the RMPE levels of citrate. FIG. 3G shows the ATMPE levels of the metabolites involved in oxidative protection. FIG. 3H shows ECAR and OCR analysis of hMSC metabolic phenotype during culture expansion. Proteomics analysis of hMSCs at early passage and late passage: FIG. 3I shows that differentially expressed proteins and (3J) PCA clustering analysis of hMSCs at P4, P8 and P12. FIG. 3K shows gene ontology (GO) analysis for proteins in biological process, cellular component, and molecular function. FIG. 3L shows Kyoto Encyclopedia of Genes and Genomes (KEGG) analysis to identify the key pathway enrichment. FIG. 3M shows Ingenuity Pathway Analysis (IPA) analysis to identify the key pathways. *, p<0.05; **, p<0.01. FIG. 3N shows OCR and ECAR in hMSCs at P5 and P12, determined by seahorse flux analyzer. (A) OCR measurement of hMSCs at baseline and after stressed by oligomycin/FCCP. (B) ECAR measurement of hMSCs at baseline and after stressed by oligomycin/FCCP.

FIGS. 4A, 4B, 4C, 4D, 4E, 4F, 4G, 4H, and 4I show the NAD+/NADH-Sirt axis imbalance induced by in vitro culture expansion of hMSCs. FIG. 4A shows NAD+ and NADH levels alteration during culture expansion of hMSCs. FIG. 4B shows NAD+/NADH ratio decreased during culture expansion. FIG. 4C show immunocytochemistry of Sirt-1 staining in culture expanded hMSCs. FIG. 4D shows Sirt-1 and (4E) Sirt-3 protein levels characterized by flow cytometry. Gene expression of (4F) Sirt-1 and Sirt-3, (4G) DNA repair and mitochondrial regulation (PARP-1, PGC-1 and TFAM), and (4H) FOXO (FOXO-1 and FOXO-3) determined by RT-PCR. FIG. 4I shows Sirt-1, Sirt-3, PGC-1α protein levels during culture expansion of hMSCs characterized by western blot. Scale bar is 100 μm. *, p<0.05; **, p<0.01.

FIGS. 5A, 5B, 5C, 5D, and 5E show in vitro culture expansion of hMSCs induced changes in NAD+ biogenesis and consumption. FIG. 5a shows mRNA levels of genes in NADase-2 activity. NAD+ metabolic enzymes including (5B) NAMPT, (5C) CD38 and (5D) CD73 was increased in late passage of hMSCs determined by flow cytometry. FIG. 5E shows confirmation of CD38, CD73, and Nampt protein expression by western blot. *, p<0.05.

FIGS. 6A, 6B, 6C, 6D, 6E, 6F, 6G, 6H, 6I, 6J, 6K, 6L, 6M, and 6N show NAD+ repletion restores mitochondrial function and preserves stem cell function in long-term cultured hMSCs. FIG. 6A shows NAD+ and NADH levels of senescent hMSCs with NAM treatment. FIG. 6B shows NAD+ and NADH ratio was increased in senescent hMSCs with NAM treatment, as well as Sirt-1/3 expression (6C). FIG. 6D shows SA-β-gal activity was decreased in senescent hMSCs with NAM treatment. FIG. 6E shows colony forming ability, and glycolytic ATP ratio (6F) were also improved in senescent hMSCs with NAM treatment. FIG. 6G shows basal autophagy was restored in senescent hMSCs with NAM treatment. FIGS. 6G, 6H, 6I, and 6J show mitochondrial function including mitochondrial mass (6G), total ROS (6H), ETC-I activity (6I), and membrane potential (6J) were all enhanced in senescent hMSCs with NAM treatment. FIG. 6K shows mitophagy ability was also restored in senescent hMSCs with NAM treatment. *, p<0.05; **, p<0.01. FIG. 6L shows ROS decreased in senescent hMSCs with NAM treatment. FIG. 6M shows cell cycle analysis of senescent hMSCs with NAM treatment. (A) hMSCs at P12. (B) hMSCs at P12 with 96 hr treatment of NAM. FIG. 6N shows nuclear magnetic resonance (NMR) spectrum of human mesenchymal stem cell (hMSC) culture medium, NAD⁺, NADH, and NAD⁺ precursor NAM. NMR spectra of different samples were obtained on a Bruker 500M spectrometer at 200 MHz. The spectrum was taken in deuterated chloroform at 20° C. The peaks for fresh culture medium (green), NAD⁺ (yellow), NADH (red), and NAM (black) were labeled in the spectra.

FIGS. 7A, 7B, 7C, 7D, 7E, 7F, 7G, 7H, 7I, and 7J show replicative culture expansion of human dermal fibroblasts (hFBs) exhibited limited cellular senescence, mitochondrial alteration and NAD+ decline. FIG. 7A shows population doubling time. FIG. 7b shows beta-gal activity for culture expanded hMSCs. FIG. 7C shows NAD+ and NADH levels and NAD+/NADH ratios during culture expansion of hFBs. FIG. 7D shows Sirt-1 and (7E) Sirt-3 protein expression determined by flow cytometry. FIG. 7F shows mitochondrial mass and (7G) membrane potential indicated no difference during culture expansion of hFBs. FIG. 7H shows autophagy and (7I) mitophagy showed no significant difference. FIG. 7J shows NAD+ metabolic enzymes Nampt, CD38 and CD73 protein expressions were comparable determined by flow cytometry.

FIG. 8 shows loss of immunomodulation in senescent hMSCs during culture expansion. In detail, IDO activity is significantly decrease in P12 hMSCs. Inflammation regulator NF-kB and COX2 expressions are increased in senescent hMSCs. Moreover, immune-cytokine profile is altered during culture expansion (increase in pro-inflammation and decrease in anti-inflammation)

FIG. 9 shows hMSCs are cultured in vitro for expansion purpose, with the increase of senescent population and decline of stem cell properties. Cellular compartments are dysfunctional during replicative expansion, leading to imbalance of energy and cellular homeostasis.

FIGS. 10A, 10B, 10C, 10D, 10E, 10F, 10G, and 10H show culture induced hMSC senescence and function decline. FIG. 10A shows hMSC morphology and size alteration during in vitro culture expansion; (10B) population doubling time, (10C) colony forming ability and (10D) cellular senescence of hMSCs at different passages; (10E) Stem cell gene and (10F) cell cycle gene of hMSCs at different passage; (10G) Total reactive oxygen species (ROS) and (10H) mitochondrial ROS of hMSCs at different passages.

FIGS. 11A, 11B, 11C, 11D, 11E, and 11F show metabolic reconfiguration in replicative cultured hMSCs. FIG. 11A shows Glycolytic ATP of passage-dependent hMSCs; (11B) Extracellular acidification rate(ECAR), (11C) oxygen consumption rate (OCR), (11D) OCR/ECAR and (11E) metabolic potential of hMSCs at different passage; (11F) GC-MS analysis of metabolites involved in glycolysis and TCA cycle, shown as absolute molar percent enrichments (ATMPE) and relative molar percent enrichments (RMPE).

FIGS. 12A, 12B, 12C, 12D, 12E, 12F, and 12G show mitochondrial function decline in replicative expanded hMSCs. FIG. 12A shows mitochondrial morphology change, (12B) mitochondrial fusion and fission gene, (12C) mitophagy and (12D) autophagy, (12E) mitophagy and autophagy gene in hMSCs at different passages; (12F) mitochondrial mass and (12G) membrane potential hMSCs from different passages.

FIGS. 13A, 13B, 13C, and 13D show Immuno-staining of Sirt-1 of hMSCs at different passages NAD+/NADH redox cycle was imbalanced in long-term cultured hMSCs. FIG. 13A shows intracellular NAD+ and NADH level and (13B) NAD+/NADH ratio in hMSCs at different passages; (13C) Gene expression of Sirt-1 and Sirt-3 in hMSCs at different passages; (13D) Sirt-1 expression in hMSCs via ICC staining.

FIG. 14 shows the mechanism of how NAD+/NADH and mitochondria regulate hMSCs cellular homeostasis during in vitro culture expansion. Culture expansion of hMSCs results in accumulation of DNA damage, which further activates PARP signal and causes the intracellular NAD+ decrease. Imbalanced NAD+/NADH level causes NAD+ dependent Sirtuin inactivation, which down-regulates several pathways, including mitochondrial biogenesis, anti-oxydant protection, and immunomodulation. Dysfunction of mitochondria further accumulates NADH and consumes NAD+ to maintain cellular function, leading energy metabolism shift from glycolysis towards OXPHOS. Maintaining intracellular NAD+ pool size via supplement of NAD+ precursors could enhance hMSCs resistance to senescence during replicative expansion.

FIGS. 15A, 15B, 15C, 15D, 15E, 15F, 15G, 15H, 15I, and 15J show the rejuvenation of hMSC cellular homeostasis via changing intracellular NAD+ level. FIG. 15A shows NAD+ and NADH level and (15B) NAD+NADH ratio after supplement of NAD+ precursor in culture induced senescent hMSCs; (15C) CFU ability and (15D) cellular senescence of high passage hMSCs with supplement; (15E) mitochondrial membrane potential and (15F) mass in high passage hMSCs with supplement; (15G) Glycolysis, (15H) mitochondrial ETC activity, (15I) autophagy and (15J) mitophagy were also enhanced by NAD+ precursor supplement in culture induced senescent hMSCs.

FIG. 16 shows a comparison of the effects of various NAD+ precursors on stem cells in culture. In detail, NAM and NR have no significant difference in boosting intracellular NAD+ level and RAD+/NADH ratio. However, NAM is better in boosting intracellular NAD+ level compared to NMN and this has been tested throughout the rejuvenation of cellular senescence, glycolytic activity and mitochondrial fitness.

FIG. 17 shows that a continuous supplement of NAD+ precursor NAM (1 mM) maintains hMSC cellular NAD+ level and Sirt activity, as well as mitochondrial fitness.

FIG. 18 shows the decreased immunomodulatory potentials of senescent hMSCs with significantly higher pro-inflammation.

FIG. 19 shows that NAD+ supplement restores hMSC immunomodulation with potent anti-inflammation.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “administering” refers to an administration that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. Embodiments defined by each of these transition terms are within the scope of this invention.

An “effective amount” is an amount sufficient to effect beneficial or desired results. An effective amount can be administered in one or more administrations, applications or dosages.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF METHODS OF CULTURING AND/OR EXPANDING CELL OR CELL LINE

In the past decades, human mesenchymal stem/stromal cells (hMSCs) have become attractive candidate for cell therapy, in the context of multilinage differentiation, paracrine effect, and immunomodulation. For clinical applications, preparation of hMSCs has to meet high-dosage requirement in industrial manufacturing as translational and therapeutic products. Large-scale production of hMSC represents the first major effort to expand adherent cells as transfusion therapeutics in cell therapy with promising resulting cell numbers. However, inconsistency of preclinical results of hMSCs makes its further elevated application unsure, possible due to donor age or morbidity, isolation difference and extensive in vitro culture expansion. hMSCs from aged or disease donor exhibit reduced stemness and altered therapeutic efficacy including paracrine and immunomodulatory functions. Studies have shown culture-induced decline of hMSC functional properties under artificial environment. Expanded hMSC passaging reduces CFU-F and proliferation rate corresponding with increased senescence and enlarged cell size. Extended passaging of hMSC also reduced therapeutic potency in preclinical and clinical studies. Although the increased senescent population and altered secretory profile have been postulated as the major factor, the mechanism underpins the culture-induced senescence in hMSC is not well understood.

In its nature, hMSCs exert heterogeneity not only at phenotype level, but also at primary metabolic state. Upon isolation of hMSCs from in vivo niche for expansion, in vitro nutrient rich environment induces rapid cell proliferation which requires energy and anabolic macromolecules for daughter cell replication. Catabolic and anabolic pathways are interconnected and together play active role in providing energetic sources and macromolecules to maintain cellular homeostasis. Under this context, hMSCs exhibit metabolic plasticity and can adopt their metabolic profile towards efficient oxidative phosphorylation (OXPHOS), that is drastically different from their in vivo quiescent glycolysis. Beyond a role in energetic support, metabolic circuits engage master genetic programs, and intermediate metabolites mediate cell signaling and regulatory pathways. Thus, metabolic plasticity allows hMSCs to match divergent demand of stem cell properties including self-renewal and differentiation. Specific connections between hMSC phenotype and metabolism are also illustrated. However, prior to the present disclosure, detailed metabolic profile and alterations that regulate hMSC fate during culture expansion are yet to be identified.

As hMSCs utilized both glycolysis and OXPHOS (specifically, TCA cycle) to proliferate and drive other cellular events, intermediate metabolites can play a role in specific singling pathways or events. NAD+/NADH redox cycle has been shown to participate energy metabolism extensively including glycolysis, pyruvate dehydrogenase complex, tricarboxylic acid (TCA) cycle and OXPHOS. NAD+(the oxidized form of nicotinamide adenine dinucleotide), as an intermediate metabolite, is also a co-substrate for enzymes such as Sirtuins that regulate cellular homeostasis and lifespan of organism, connecting energy metabolism to cellular homeostasis. Sirtuins (Sirt-1 as best-characterized in mammalian) are enzyme family utilizing NAD+ as co-substrates to catalyze the deacetylation of histones in target proteins involved in aging process, including P53, PARP, FOXOs, nuclear factor-kB (NF-kB), PGC-1a, and Ku70. Sirtuin-reaction is NAD+ dependent and thus makes NAD+a rate-limiting substrate in these pathways. Alteration of intracellular NAD+ level has been shown to influence cellular metabolism along with reduced sirtuin activity. Besides, other signaling pathways in non-redox reactions also consume NAD+ to activate downstream functions, such as ADP-ribosyltransferases, including poly(ADP-ribose) polymerases (PARPs) and cyclic ADP-ribose synthases (cADPRSs) including CD38/CD157, which appear to be the major enzymes to reduce NAD+ level. Increasing body of evidence in recent studies indicated that decreasing intracellular NAD+ level and Sirt-1 activity is associated with metabolic diseases and aging. Strategies targeting NAD metabolism on majoring pathways in cells, such as NAMPT, CD38, and CD73 demonstrate promising results to perturb NAD+ biosynthesis and consumption. Yet, loss of NAD+/NADH balance along with aging is still under investigation. Moreover, prior to the present disclosure, the role of NAD/NADH redox balance and sirt-1 function in hMSC with culture-induced senescence under in vitro expansion is yet to be studied and understood.

As is shown herein, restoring NAD+ level in a cell can reduce and/or inhibit the cellular events leading to cultural induced senescence. Accordingly, in one aspect, disclosed herein are methods of culturing and/or expanding a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts) said method comprising placing cell or cell line in a media comprising a nicotinamide adenine dinucleotide (NAD+) precursor (such as, for example, nicotinamide (NAM), nicotinamide mononucleotide (NMN), and/or nicotinamide riboside (NR). It is understood and herein contemplated that by adding a NAD+ precursor (such as, for example NAM, NMN, and/or NR) cell culture induced senescence is prevented, inhibited, and/or reduced. Thus, disclosed herein are methods of preventing, inhibiting, and/or reducing cell culture induced senescence of a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts) said method comprising placing cell or cell line in a media comprising a nicotinamide adenine dinucleotide (NAD+) precursor (such as, for example, nicotinamide (NAM), nicotinamide mononucleotide (NMN), and/or nicotinamide riboside (NR)). In one aspect, the media does not need to comprise the NAD+ precursor, but the NAD+ precursor can be added to the media before or after the addition of the cell or cell lines.

In one aspect, the NAD+ precursor (such as, for example, NAM, NMN, and/or NR) can be added to the media prior to adding the cells for culturing and/or expansion. In one aspect, the NAD+ precursor (such as, for example, NAM, NMN, and/or NR) can be added after or concurrently with the addition of the cells or cell line(s) for culturing and/or expansion, but prior to the potential for any culture induced senescence. For example, the NAD+ precursor can be added during the initial culture or any passage after that including, but not limited to passage (P) 1 (P1), P2, P3, P4, P5, P6, P7, P8, P9, P10, P11, P12, P13, P14, P15, P16, P17, P18, P19, P20, P21, P22, P23, P24, P25, P26, P27, P28, P29, P30, P35, P40, P45, P50, P55, P60, P65, P70, P75, P80, P85, P90, P95, or P100. In one aspect, the NAD+ precursor (such as, for example, NAM, NMN, and/or NR) is added to the culture at a passage closer when culture induced senescence is typically observed in the cell or cell line being propagated. In one aspect, the NAD+ precursor is added after P5, P6, P7, P8, P9, P10, P11, P12, P13, P14, P15, P16, P17, P18, P19, P20, P21, P22, P23, P24, P25, P26, P27, P28, P29, P30, P35, P40, P45, P50, P55, P60, P65, P70, P75, P80, P85, P90, P95, or P100.

However, it is also understood and herein contemplated that the effects that the addition of NAD+ precursors has on the cells to inhibit, reduces, and/or prevent culture induced senescence can also have a therapeutic effect on a cell culture already showing indications of senescence or having become senescent. Accordingly, disclosed herein are methods of rescuing a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts) from senescence during culture and expansion said method comprising placing cell or cell line in a media comprising a nicotinamide adenine dinucleotide (NAD+) precursor (such as, for example, nicotinamide (NAM), nicotinamide mononucleotide (NMN), and/or nicotinamide riboside (NR)) or adding said precursor to the culture media before or after the addition of the cells or cell lines. It is understood and herein contemplated that to rescue a cell or cell line for culture induced senescence, the cell or cell line must already have become senescent or showing indication of becoming senescent. Thus, in one aspect, the NAD+ precursor (such as, for example, NAM, NMN, and/or NR) to the culture media after culture induced senescence has been detected. As with the methods of culturing and/or expanding cell or cell line and methods of preventing, inhibiting, and/or reducing cell culture induced senescence of a cell or cell line, for the disclosed methods of rescuing a cell or cell line from senescence during culture and expansion, the NAD+ precursors can be added at any passage once signs of senescence have commenced. For example, the NAD+ precursor can be added following passage (P) 1 (P1), P2, P3, P4, P5, P6, P7, P8, P9, P10, P11, P12, P13, P14, P15, P16, P17, P18, P19, P20, P21, P22, P23, P24, P25, P26, P27, P28, P29, P30, P35, P40, P45, P50, P55, P60, P65, P70, P75, P80, P85, P90, P95, or P100.

Passaging can take place at a rate sufficient to have cells reach the desired level of confluency or expansion in the culture media (including attachment time where appropriate) before the culture must be transferred to a new culture vessel (i.e., passaged). The amount of time required for the cells to have obtained the appropriate degree of expansion can depend on the cell type, media, culture vessel size, and culture conditions. Thus, disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the cells are passaged at least once every 12, 18, 24, 30, 36, 42, 48, 54, 60, 66, 72, 78, 84, 90, 96, 102, 108, 114, 120, 126, 132, 138, 144, 150, 156, 162, or 168 hours.

Once administration of the NAD+ precursor begins, the NAD+ precursors can be added at a rate effective to reduce, inhibit, or prevent senescence or to rescue the cell or cell line from senescence. For example, the NAD+ precursor (such as, for example, NAM, NMN, and/or NR) can be added at least once every 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 passages. In some instances that rate administration of the NAD+ precursor (such as, for example, NAM, NMN, and/or NR) can be different for rescue and subsequence culture to prevent, inhibit, and/or reduce future senescence once the cell or cell line has been rescued. In other words, there can be a first rate of administration and a second rate of administration. For example, disclosed herein are methods of methods of rescuing a cell or cell line from senescence during culture and expansion comprising adding NAD+ precursors (or media comprising said precursors) to a cell culture at a first administration rate of at least once every 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 and then, once rescued continuing to culture and/or expand the cells or cell lines comprising placing cell or cell line in a media comprising a nicotinamide adenine dinucleotide (NAD+) precursor (such as, for example, nicotinamide (NAM), nicotinamide mononucleotide (NMN), and/or nicotinamide riboside (NR)) or adding said precursor to the culture media before or after the addition of the cells or cell lines, wherein NAD+ precursors (or media comprising said precursors) to a cell culture at a second administration rate of at least once every 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 passages thereafter, wherein the rate of administration is faster, slower, or the same as the first rate of administration, but is independent of the first rate of administration.

It is further understood and herein contemplated that there are situations where the time between passages is such that a single application of NAD+ precursor (such as, for example, nicotinamide (NAM), nicotinamide mononucleotide (NMN), and/or nicotinamide riboside (NR)) is not sufficient to maintain effective amounts or concentrations of NAD+ precursors. Thus, in one aspect, disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the NAD precursor is added to the culture at least one time every 12, 18, 24, 30, 36, 42, 48, 54, 60, 66, 72, 78, 84, 90, 96, hours, 5, 6, 7, 8, 9, 10, 11, 12, 13, 14, 15, 16, 17, 18, 19, 20, 21, 22, 23, 24, 25, 26, 27, 28, 29, 30, 31, 60 days. For example, disclosed herein are methods of culturing and/or expanding cell or cell line; preventing, inhibiting, and/or reducing cell culture induced senescence of a cell or cell line; and/or rescuing a cell or cell line from senescence during culture and expansion of any preceding aspect, wherein the NAD precursor is added to the culture at least 1, 2, 3, 4, 5, 6, 7, 8, 9, 10, 11, 12, 13, or 14 times per week. In some aspects, the NAD precursor is added to the culture at least 1, 2, 3, 4, 5, 6, 7, 8, 9, 10, 11, 12, 13, or 14 times per passage. In one aspect, the appropriate concentration of NAM, NMN, and/or NR to prevent, inhibit, reduce, or rescue culture induced senescence can be between 0.1 mM and 100 mM, preferably between 1 mM and 20 mM. For example, the concentration of NAM, NMN, and/or NR when added to the media can be 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 mM.

In some aspects, senescence, pluripotency, mitochondrial fitness, metabolism, cellular homeostasis, NAD+ Sirt axis, immunomodulation, and/or therapeutic effect is assessed before NAD+ precursors are added to a media or media comprising NAD+ precursors are used to culture the cells or cell lines.

It is understood and herein contemplated that the disclosed methods involve culturing cells and that the particular culture conditions such as the presence of serum, media supplements, CO₂ percentage, temperature, O₂ percentage, and/or shaking can vary between cell types. In some aspects, it is specifically understood that the Oxygen tension of the culture is NOT reduced. In some aspect glycolysis of the culture is enhanced.

It is understood that the methods of culturing and/or expanding cell or cell line; preventing, inhibiting, and/or reducing cell culture induced senescence of a cell or cell line; and/or rescuing a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts) from senescence during culture and expansion can be effective with any cell or cell line where culture induced senescence has been observed, including, but not limited to a stem cell or stem cell cell line, such as, for example, human mesenchymal stem cells or cell lines (hMSC), bone marrow mesenchymal stem cells or cell lines, and fibroblast cell s or cell lines.

The disclosed methods involve supplementing culture media with NAD+ precursors (such as, for example, nicotinamide (NAM), nicotinamide mononucleotide (NMN), and/or nicotinamide riboside (NR)) to prevent, inhibit, reduce, and/or rescue cells or cell lines from culture induced senescence. In one aspect, also disclosed herein is culture media comprising one or more NAD precursors (such as, for example, NAM, NMN, and/or NR). In one aspect, the media used in any of the disclosed methods or to which NAD+ precursors are added can be any media known in the art appropriate for growing and expanding the disclosed cells, for example, the media can comprise Roswell Park Memorial Institute (RPMI) 1640, complete culture medium (CCM), Minimal Essential Medium (MEM), Dulbecco's Modified Eagle Medium (DMEM), Iscove's Modified Dulbecco's Medium (IMDM), Eagle's mimumal essential medium (EMEM), Cell Therapy systems (CTS) essential 8 medium, Medium 199, essential 8 medium, StemFlex medium, and AdvanceSTEM cell culture media. In some aspects, the culture media can comprise a Xeno-free culture medium.

Disclosed herein are media comprising NAD+ precursors and methods of culturing and/or expanding cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts); preventing, inhibiting, and/or reducing cell culture induced senescence of a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts); and/or rescuing a cell or cell line (such as, for example a stem cell or stem cell cell line including but not limited to human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts) from senescence during culture and expansion comprising adding cells to said media or supplementing media with NAD+ precursors during culture/expansion of the cells. It is understood and herein contemplated that the NAD+ precursors used in said media and/or methods can comprise any NAD+ precursor that has the desired effect of restoring NAD+ levels such that culture induced senescence is prevented, reduced, inhibited, or rescued. As used herein NAD+ precursors include, but are not limited to nicotinamide (NAM), nicotinamide mononucleotide (NMN), and/or nicotinamide riboside (NR).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Metabolism and NAD+/NADH Redox Cycle in hMSCs Homeostasis and Rejuvenation During Culture Expansion

Considering the evidence of hMSC acquire metabolic plasticity and the role of NAD+/NADH in metabolism, the hypothesis that hMSC under in vitro expansion exhibit metabolic shift from aerobic glycolysis to OXPHOS and a corresponding decline in intracellular NAD+ level was tested, resulting in reduced Sirt-1 signaling and a breakdown of cellular homeostasis. Repletion of NAD+ in senescent hMSCs recovers mitochondrial fitness and stem cell properties. Moreover, it was also shown that stem cells with definite life span and ability in senescence exhibited different NAD+ metabolism compare to other primary cells such as fibroblast. Together, the results reveal a novel metabolic and biochemical marker in hMSC fate which can be used to evaluate hMSC cellular homeostasis and stem cell quality in biomanufacturing.

### a) Results

(1) hMSCs Exhibit Senescence and Functional Decline During In Vitro Replicative Expansion.

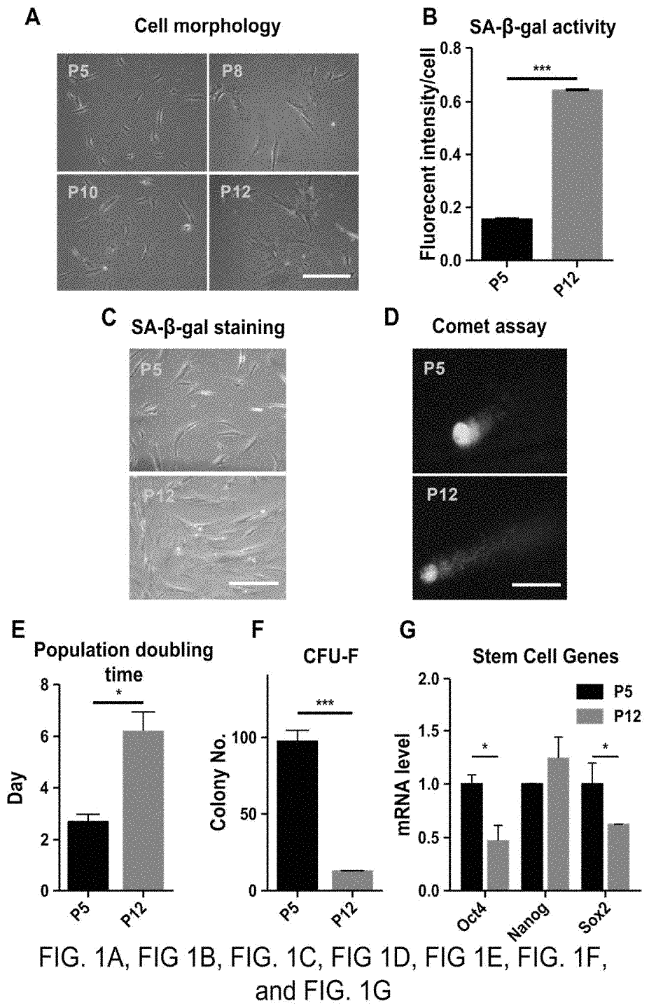

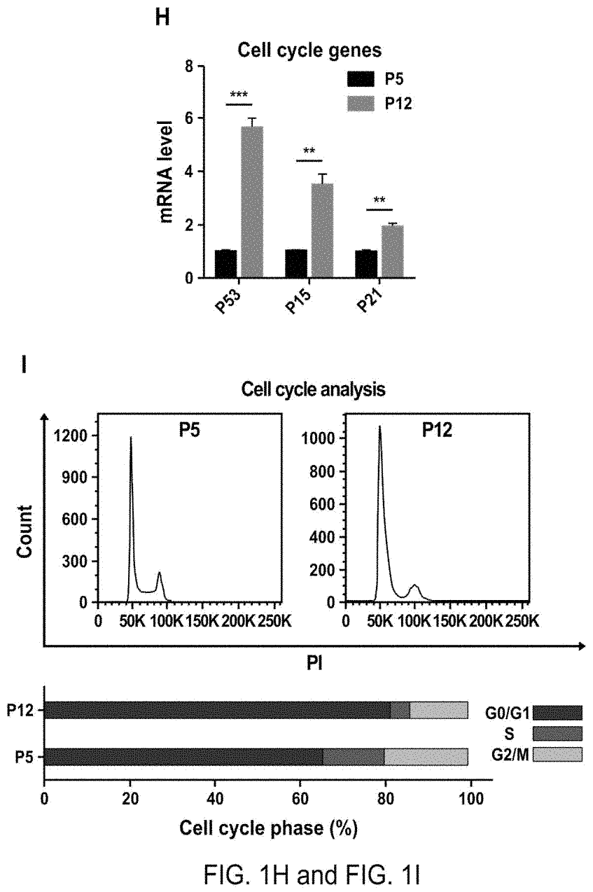

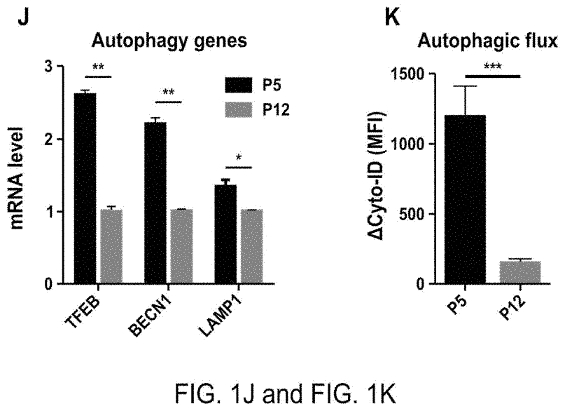

Culture-expansion of hMSCs in vitro is essential for application of hMSCs. The isolation and culture condition for MSC expansion follows the most widely applied method with standardized medium. hMSCs exhibited significant morphological change from spindle-shape to flat-shape along with enlarged cell size during in vitro expansion from early passage (P5) to late passage (P12) (FIG. 1A). Generally, enlarged cell size indicates hMSC senescence, which was also evaluated by the increasing SA-β-gal activity and staining (FIGS. 1B and C). DNA damage was observed in late passage (passage 14) of hMSCs with senescence via comet assay (FIG. 1D). hMSCs at passage 5 exhibited less tail/body length compared to those at passage 12, indicating less DNA damage. hMSCs with replicative senescence exhibited increased population doubling time (6 days of P12 vs. 2.8 days of P5) (FIG. 1E). At the same time, senescent hMSCs lost their self-renewal ability characterized by CFU-F assay, as colony number decreased from 96 (P5) to 10 (P12) (FIG. 1F). Stem cell genes Oct4 and Sox2 were significantly down regulated in P12 hMSCs, but not Nanog (FIG. 1G). Cell cycle analysis also indicated that senescent hMSCs (passage 12) were arrested in G0/G1 phase of cell cycle, corresponding to the increased mRNA levels of cell cycle-related genes p53, p15, and p21 (FIGS. 1H and 1I). In addition, culture expansion from passage 5-6 to passage 12-13 also breaks cellular homeostasis characterized by down-regulation of autophagic genes TFEB, BECN1, and LAMP1, and inhibition of basal autophagy (FIGS. 1J and 1K). Together, these results demonstrate that in vitro culture expansion induced senescence of hMSCs with negative effects on basic cellular events and stem cell properties under standard growth conditions.

(2) Culture Expansion Induces Mitochondrial Dysfunction in Senescent hMSCs.

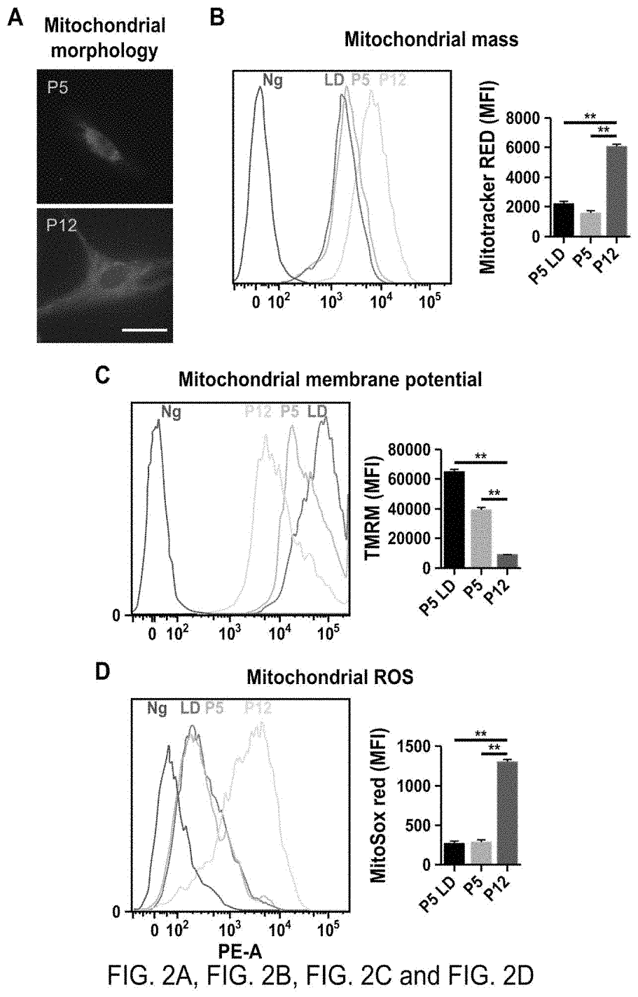

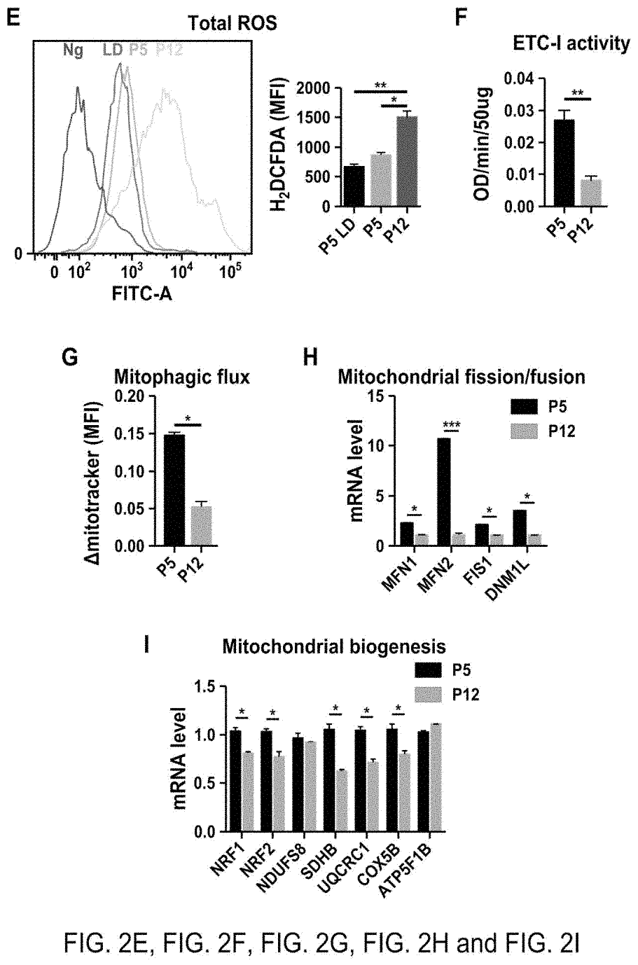

Culture expansion of hMSCs not only alters basic cellular events, but also influence the function of cellular organelle. As mitochondria plays the most important role in regulating energy homeostasis and cell function, a set of mitochondrial characteristics was screened to evaluate the fitness of hMSC mitochondria during in vitro culture expansion. Not surprisingly, mitochondrial morphology is significantly altered during culture expansion of P5 to P12, from fragmented morphology to fused-elongated shape (with circularity 0.73 vs 0.24)(FIG. 2A)). This morphological change corresponds to the increased mitochondrial mass in hMSCs at high passage (from P5 to P12) (FIG. 2B). However, mitochondrial membrane potential (MMP) is lower in late passage hMSCs (P12) compared to early passage cells (P5), indicating that membrane integrity is damaged and unable to prevent ROS leakage (FIG. 2C). The lower MMP leads to increased ROS in mitochondria and total cell body for P12 hMSCs compared to P5 hMSCs (FIGS. 2D and 2E). Moreover, electron transport chain complex I activity decreased in P12 hMSCs (FIG. 2F). Late passage of hMSCs also lose mitophagy ability compare to early passage (G). Genes (i.e., MFN1, MFN2, FIS1, and DNM1L) involved in mitochondrial fusion/fission dynamics in hMSCs at late passage (P12) was also down regulated compared to cells at low passage (P5) (FIG. 2H). Genes (i.e., NRF1, NRF2, SDHB, UQCRC1, and COXSB) involved in mitochondrial biogenesis was inhibited for P13 cells compared to P5 cells except NDUFS8 and ATP5F1B, based on the qRT-PCR results (FIG. 2I). Together, these results indicate that long term culture expansion of hMSCs impacts their mitochondrial function and biogenesis, which can contribute to senescence and the decline of cellular functions.

(3) Culture Expansion Induces Metabolic Reconfiguration in hMSCs.

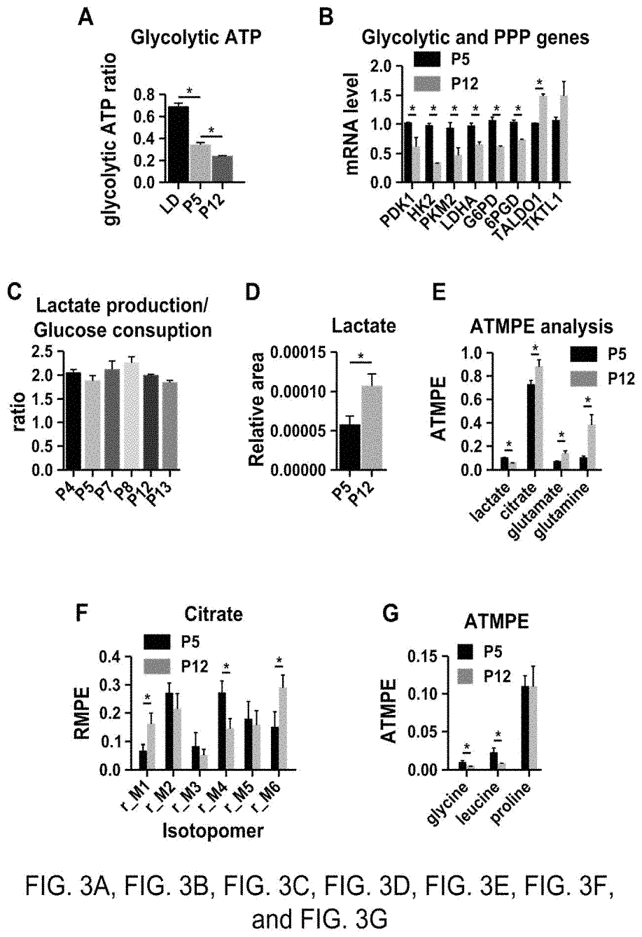

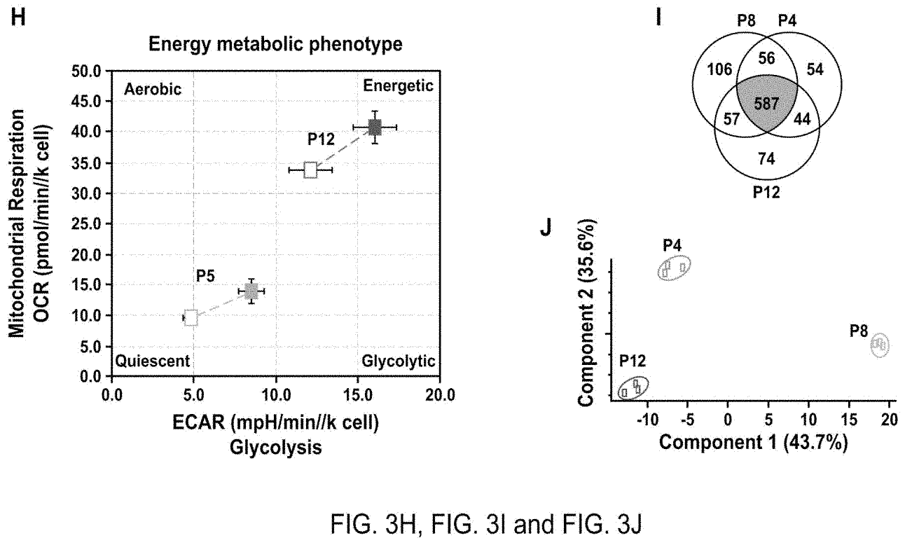

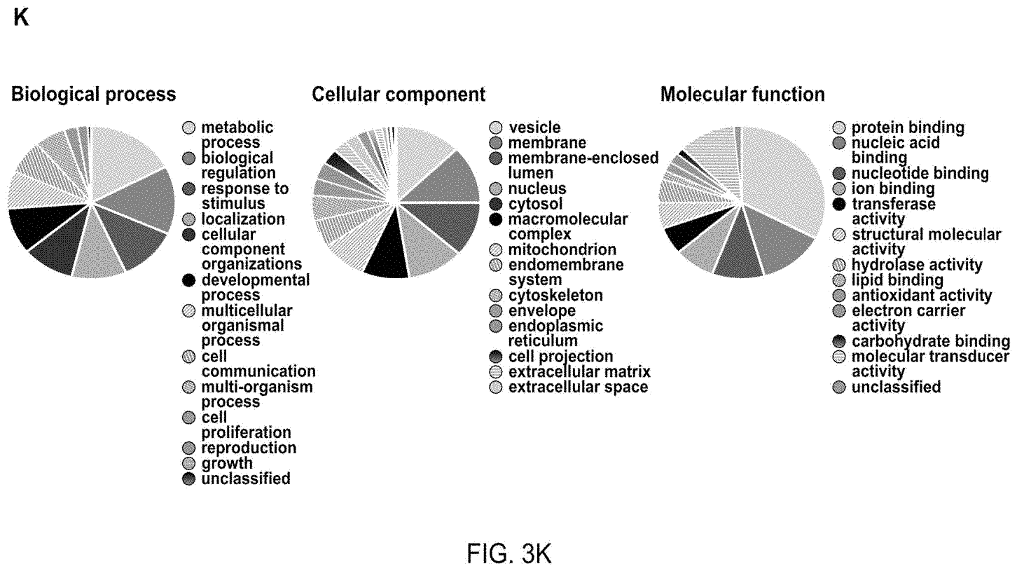

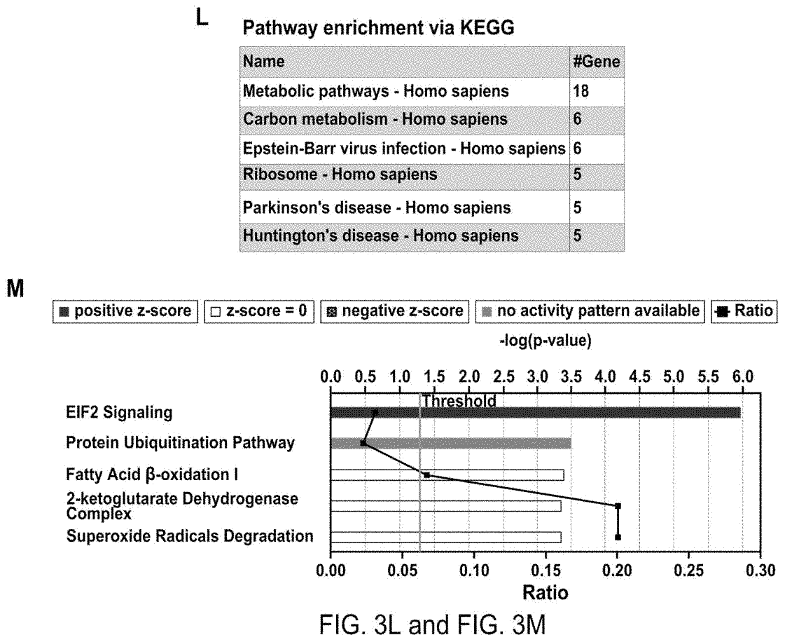

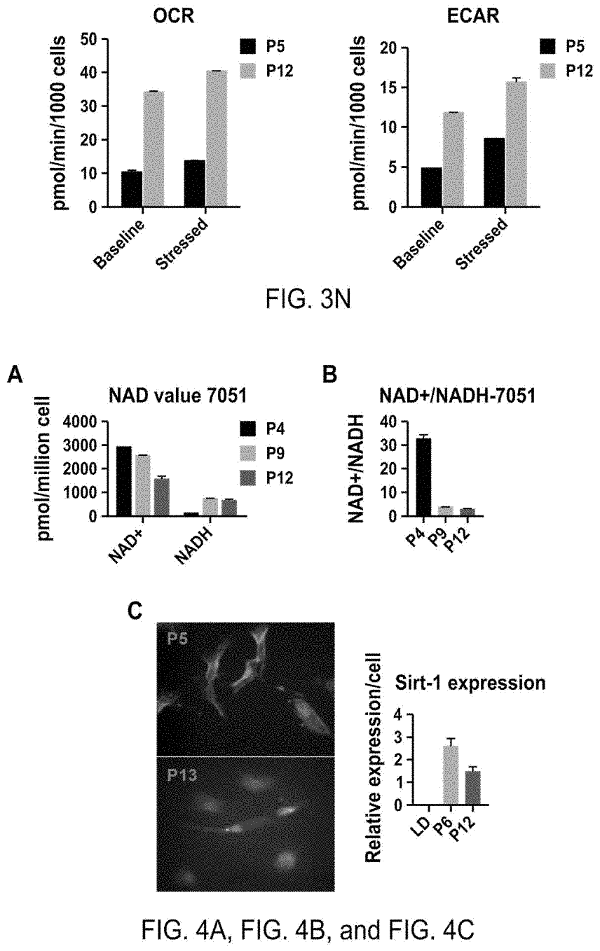

As mitochondrial plays the key role in energy metabolism, hMSC metabolic state was further characterized during culture expansion. Glycolytic ATP production was found to be significantly reduced in hMSCs at passage 12 compared to passage 5 (FIG. 3A). Genes involved in glycolysis and pentose phosphate pathway, i.e., PDK1, HK2, PKM2, LDHA, G6PD, 6PGD, were down-regulated as an indicator for metabolic reconfiguration (FIG. 3B), while TALDO1 and TKTL1 gene expression increased in late passage of hMSCs. Interestingly, the ratios of lactate production/glucose consumption are relatively stable across the expansion from P4 to P13 (FIG. 3C). Internal level of lactate increased in late passage (FIG. 3D) and ATMPE results showed the decreased lactate as well as the increased citrate in passage 12 hMSCs, indicating the closely coupled glycolytic flux with TCA cycle (FIG. 3E). However, RMPE of citrate isotopomers did not show significant difference for M2 and M3 from TCA cycle between P5 and P12 while M1, M4 and M6 are significantly changed, which can indicate other carbon source for the citrate metabolism, such as glutamate and glutamine (FIGS. 3E and 3F). Other metabolites involved in oxidative stress protection, such as glycine, leucine, and proline, were also decreased in hMSCs at passage 12 compared to cells of passage 5 (FIG. 3G). Moreover, to monitor cell metabolism in real time, the ECAR and OCR was measured via seahorse flux analyzer and the results clearly indicated the metabolic shift from glycolysis towards OXPHOS during in vitro culture expansion of hMSCs (FIG. 3H). Proteomics analysis of P4, P8 and P12 of hMSCs were performed. The results showed 587 proteins in common for the three groups (FIG. 3I). A total of 80 proteins were selected based on 2-fold change cut-off from P8 versus P4 as well as P12 versus P4. PCA analysis demonstrated a clear separation of P4, P8, and P12 samples as three replicates of each group were clustered (FIG. 3J). GO items analysis illustrated the differentially expressed proteins (DEPs): (1) primarily associated with metabolic process, biological regulation and response to stimulus in biological process category; (2) membrane and membrane-enclosed lumen in cellular component category item, while mainly associated with vesicle; and (3) principally connected to proteins associated with protein binding, nucleic acid binding and nucleotide binding in molecular function category (FIG. 3K). KEGG pathway analysis confirmed the metabolic reconfiguration during culture expansion of hMSCs (FIG. 3L). IPA analysis indicated that EIF2 signaling, protein ubiquitination, fatty acid beta-oxidation I, 2-ketoglutarate dehydrogenase complex, and superoxide radicals degradation were significantly altered in late passage of hMSCs (FIG. 3M). Among those pathways, Fatty acid β-oxidation I and 2-ketoglutarate dehydrogenase complex are associated to NAD+/NADH redox cycle and NAD+ metabolism.

(4) Culture Expansion Induces NAD+/NADH Redox Cycle Imbalance inhMSCs with Replicative Senescence.

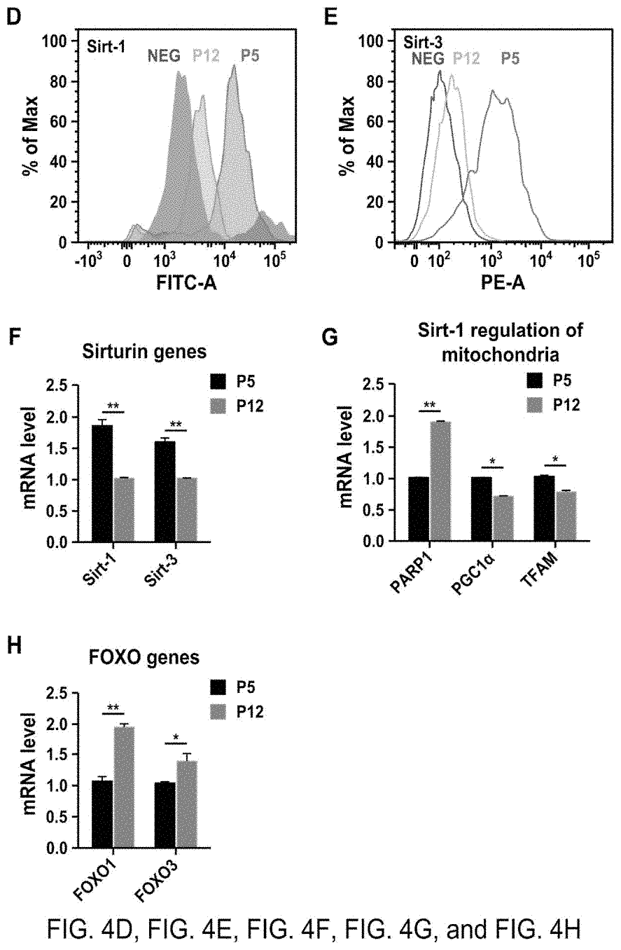

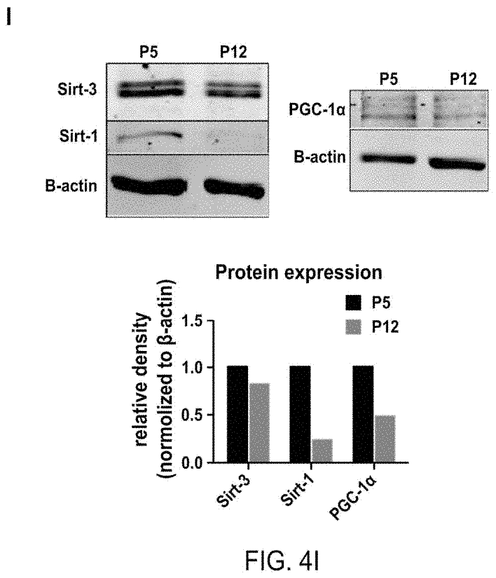

NAD+/NADH redox cycle in hMSCs connects glycolysis and TCA cycle in energy metabolism and participates in aging-related signaling pathways and functions. Since culture expansion induces metabolic reconfiguration in hMSCs, the redox cycle can also be influenced. It was found that cellular NAD+ level declines, while the reduced form of NAD+, NADH level, elevated during hMSC expansion (P4, P9, and P12), which results in the decreased ratio of NAD+/NADH (FIGS. 4A and B). As the key indicators of NAD+-dependent signaling pathway, Sirt-1 and Sirt-3 activity as well as the responding genes were also decreased in hMSCs at late passage (FIG. 4C-F). Sirt-1 regulates mitochondrial biogenesis via PGC1a and TFAM, which have been shown to decrease in P12 hMSCs, while genes involved in oxidative protection (FOXO1 and FOXO3) regulated by Sirt-1/-3 were also decreased (FIGS. 4G and H). However, PARP1 expression increased from P5 to P12 (FIG. 4G). Western blot results confirm the down-regulation of Sirt-1/-3 as well as PGC-1 at protein level from P5 to P12 (FIG. 4I).

(5) NAD+ Biogenesis and Metabolism are Altered During In Vitro Culture Expansion of hMSCs.

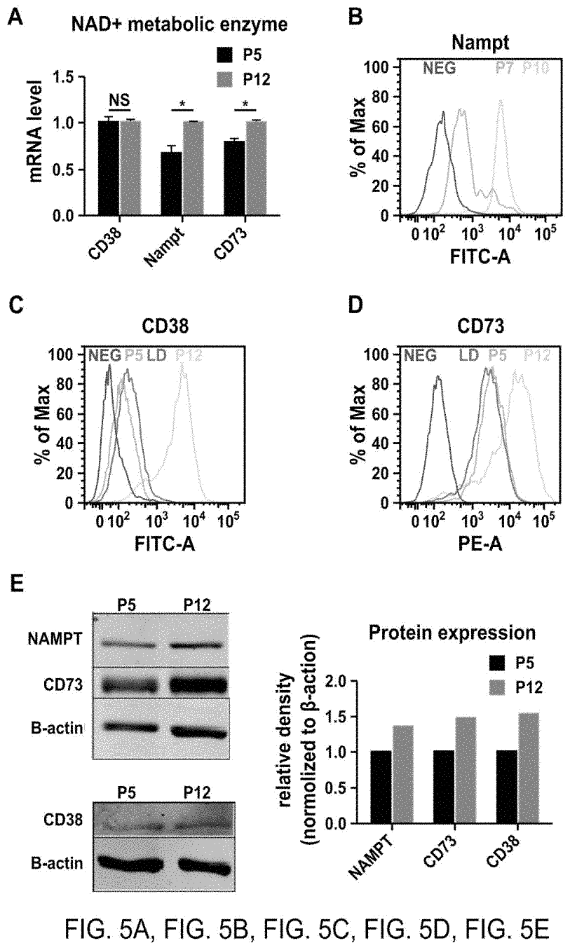

Since intracellular level of NAD+ of hMSCs declines during in vitro expansion, the major pathway of NAD+ biosynthesis and consumption was further investigated. As a rate-limiting step of salvage pathway for maintaining NAD+ level, micotinamide phosphoribosyltransferase (NAMPT) was significantly increased at late passage of hMSCs (passage 10-12) compared to hMSCs of P6-7, both at molecular level determined by RT-PCR and at protein level determined by flow cytometry (FIGS. 5A and 5B). While for the major consumption enzymes CD38 and CD73, late passage of hMSCs also exhibited higher expression compared to cells at early passage (FIGS. 5C and 5D). Western blot was performed for NAMPT, CD38, and CD73 to confirm the elevated expression during culture expansion of hMSCs (FIG. 5E). These results demonstrate culture expansion induced changes in NAD+ biosynthesis and consumption that can contribute to the imbalance of intracellular NAD+/NADH level.

(6) Re-Balancing NAD+/NADH Redox Cycle Restores Mitochondrial Fitness and Cellular Homeostasis in hMSCs with Replicative Senescence.

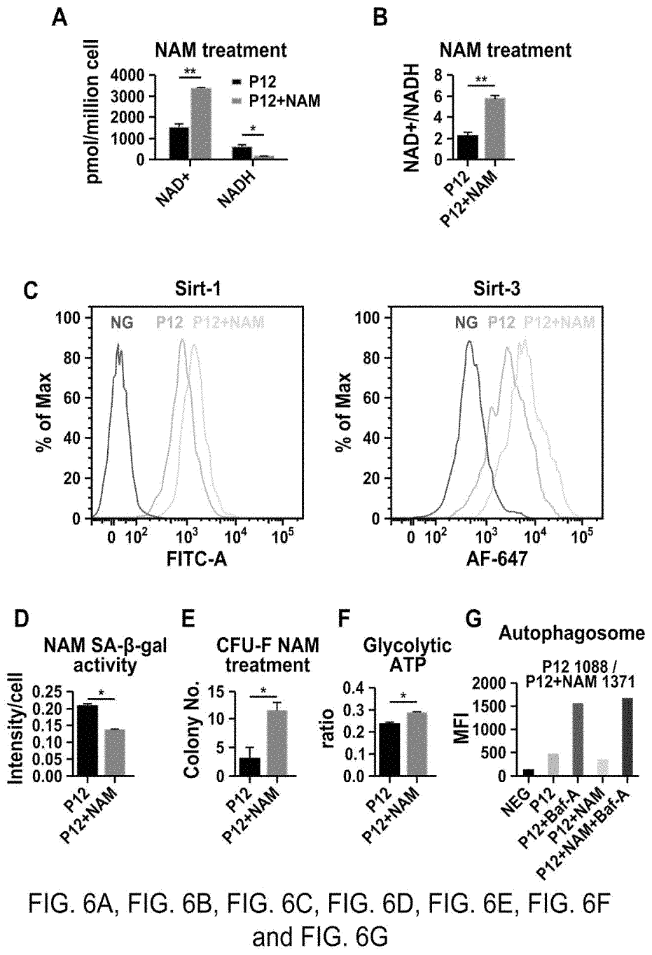

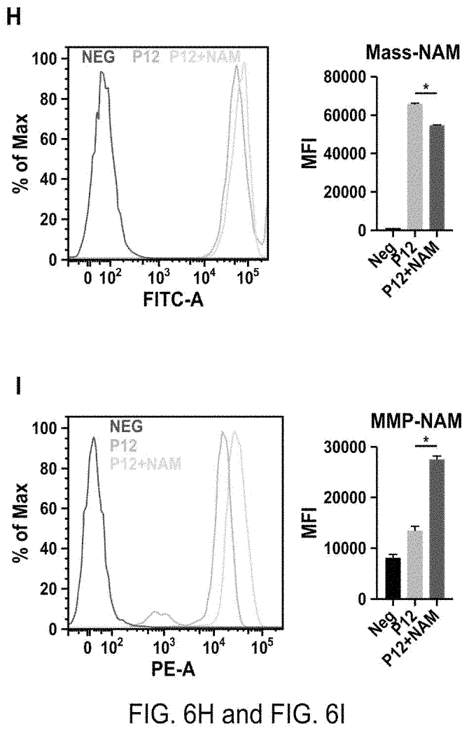

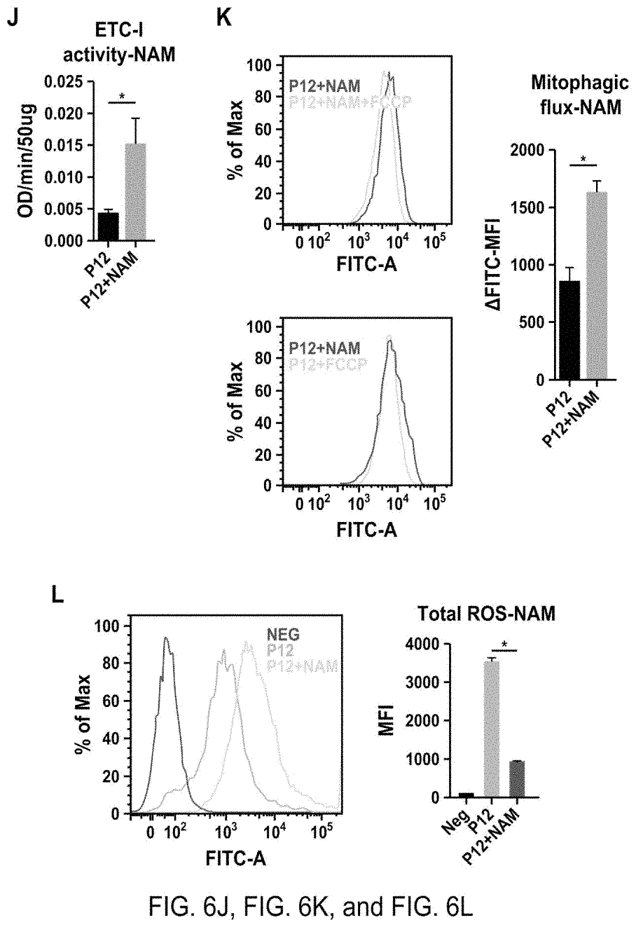

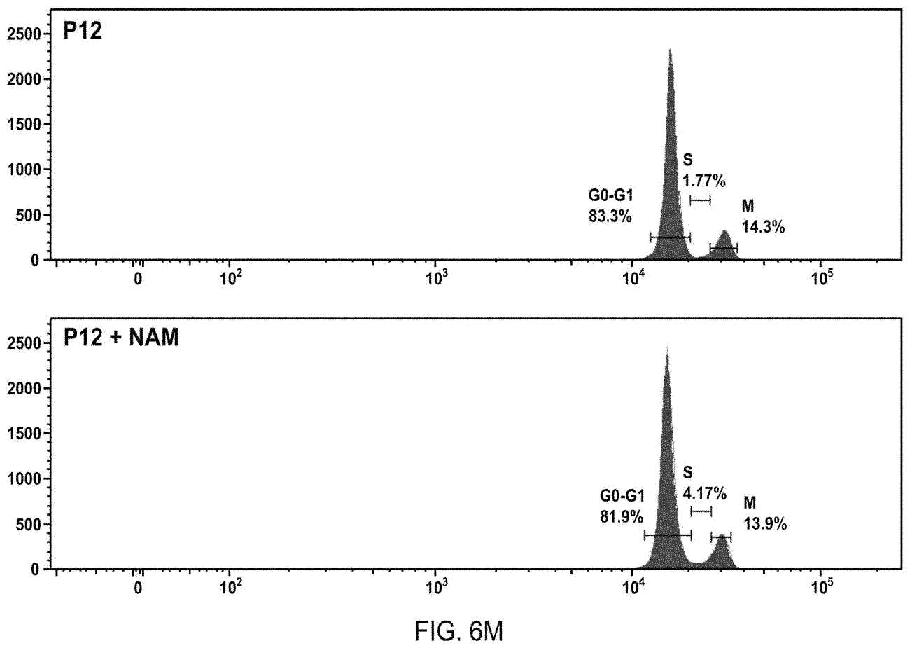

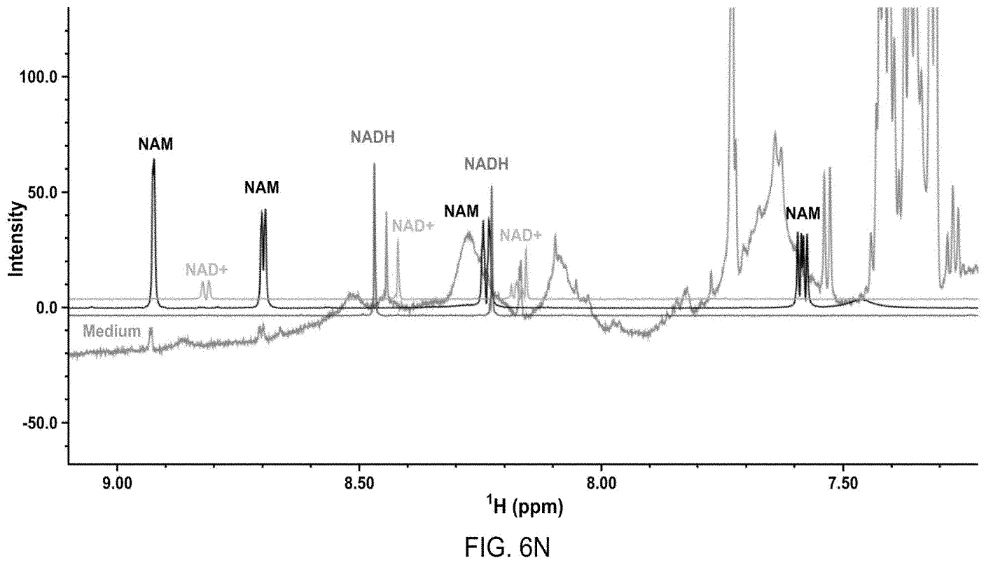

With the potential relation between NAD+ level and stem cell functions and fate, the effects of repletion of NAD+ in late passage of hMSCs with culture-induced senescence was investigated via adding NAD+ precursor NAM. Cellular NAD+ level and the ratio of NAD/NADH significantly increased after 96 h of NAM treatment (FIGS. 6A and 6B). Sirt-1 expression was also improved (FIG. 6C). hMSC senescence was also reduced, indicated by SA-O-gal activity (FIG. 6D). More importantly, colony forming ability of hMSCs (CFU-F) was restored (from 3 CFU to 11 CFU) by the NAM treatment, indicating the rejuvenation of hMSC sternness by NAD+ precursor NAM (FIG. 6E). Glycolytic ATP produced in NAM-treated senescent hMSCs was higher compare to control (FIG. 6F), which reveals the balance glycolytic metabolism. Basal autophagy level was also increased with NAM treatment (FIG. 6G). Mitochondrial fitness including mass (decreased in FIG. 6H), MMP (increased in FIG. 6I), ETC-I activity (increased in FIG. 6J), and mitophagy (increased in FIG. 6K) and total ROS (decreased in FIG. 6L), were all restored to a certain extent, indicated the recovery of mitochondrial fitness. Together, maintaining NAD+/NADH redox cycle restores mitochondrial functions and hMSC sternness, and partially reconfigure hMSC metabolism to a glycolytic phenotype.

(7) NAD+/NADH Redox Cycle and Mitochondrial Fitness are Relatively Stable During Replicative Expansion of Human Fibroblast.

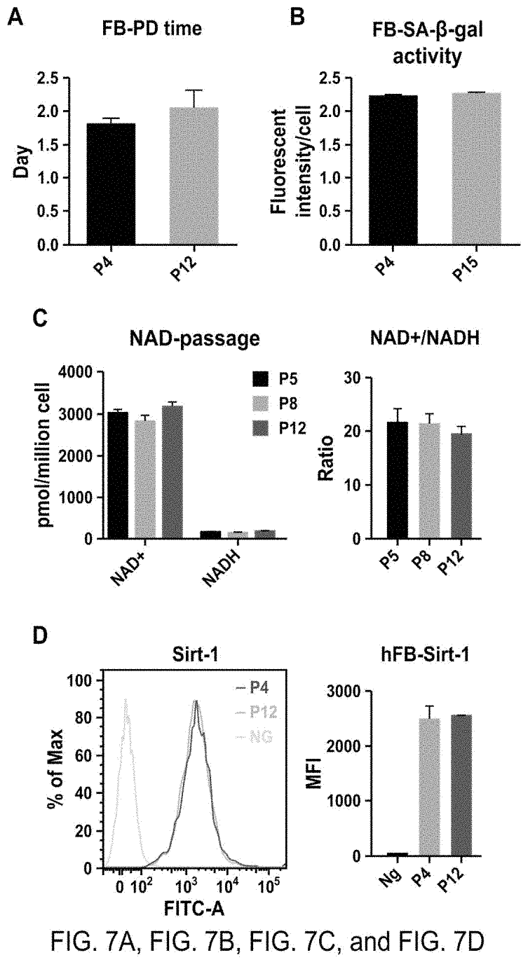

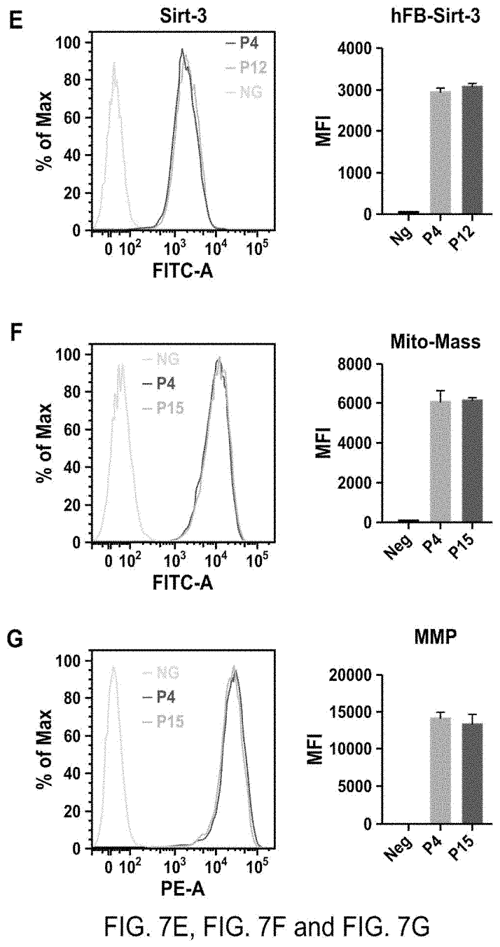

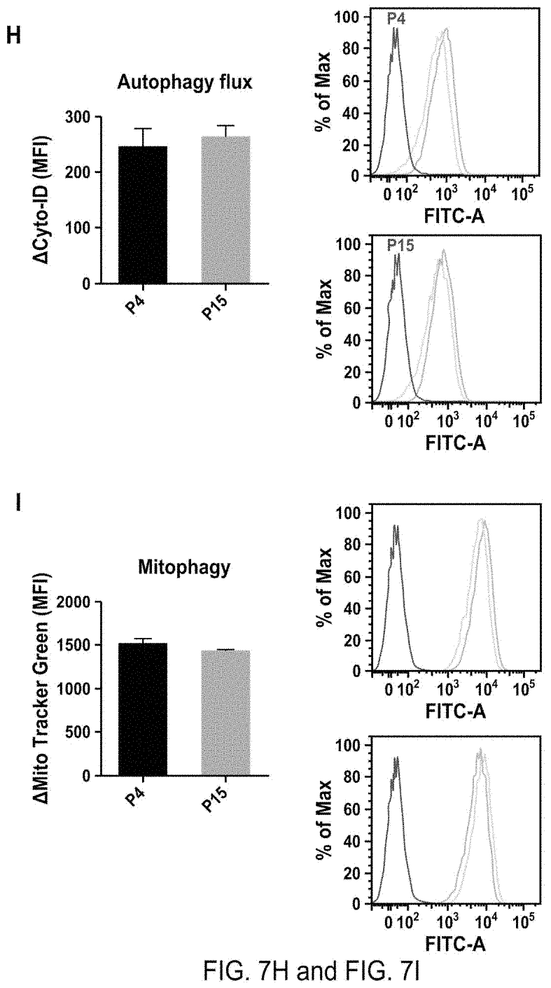

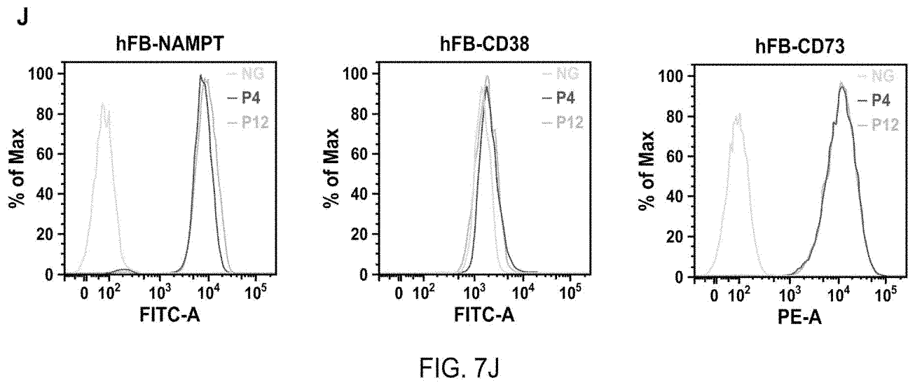

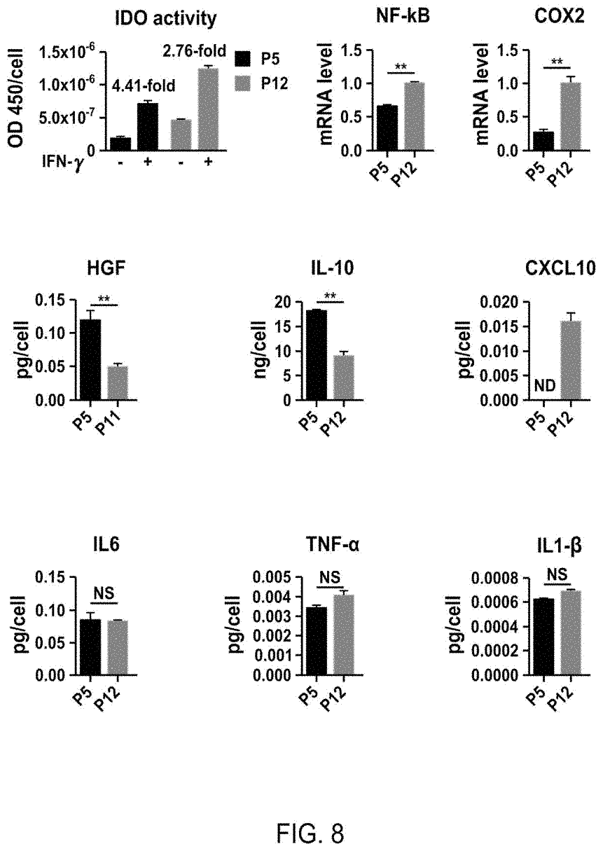

Since hMSCs exhibit significant change induced by replicative culture expansion, it was examined whether culture expansion also influence mature adult cells on their senescence and mitochondrial fitness. Here, human dermal fibroblasts were cultured up to passage 15 (P15) and their NAD+/NADH redox cycle was examined as the regulator for the mitochondrial functions. As hFBs were cultured, there was no significant difference for the population doubling time between low and high passage cells (FIG. 7A), which indicated that hFBs were not captured in cell cycle arrest during replicative expansion. As expected, cellular senescence indicated by SA-β-gal activity was also comparable between low and high passage of hFBs (FIG. 7B). More interestingly, cellular NAD+ level and NAD+/NADH ratio were not altered during culture expansion, at least within 15 passages under the same conditions as hMSCs (FIG. 7C). Also, no significant changes were found in Sirt-1 and Sirt-3 expression across the expansion of hFBs (FIGS. 7D and 7E). As shown above, hMSCs exhibit mitochondrial dysfunctions when cells are in senescence, partially due to NAD+/NADH-Sirt axis regulation. However, late passage of hFBs were shown to have similar mitochondrial activity and fitness compare to early passage cells (FIGS. 7F and 7G). Mitochondrial mass was comparable in both groups, with similar membrane potentials during extensive expansion of hFBs. More importantly, mitophagy and autophagy in high passage hFBs were not changed during the expansion, indicating the maintenance of cellular homeostasis (FIGS. 7H and 7I). Nampt, CD38, and CD73 expression was also comparable through the expansion of hFBs (FIG. 7J). Together, these data indicate that hFBs are less sensitive to replicative expansion compare to hMSCs. NAD+/NADH and sirtuin activity was well maintained to preserve cellular homeostasis and anti-senescence.

### b) Discussion

Beyond the multi-lineage differentiation, hMSCs exhibited paracrine and immunomodulatory potentials that facilitate endogenous tissue regeneration, thus are acknowledged as a potential therapeutic candidate. In order to move forward to clinical applications, in vitro large-scale expansion of hMSCs is in demand to meet clinical required cell number, with genetic stability and therapeutic efficacy. However, progressive loss of stem cell properties and genetic alterations during in vitro culture expansion of hMSCs has been widely reported as replicative senescence. In detail, loss of colony forming ability and stem cell genes in hMSCs with replicative senescence indicated the loss of their therapeutic efficacy (FIG. 1). Although DNA damage and exceed ROS accumulation have been attributed as the mechanistic driven of senescence and aging, this study provides the first connection between NAD+/NADH redox balance and cellular homeostasis, which reveal the regulatory role of metabolism and intermediate metabolites in stem cell fate.

In vitro culture conditions have been shown to significantly impact cellular behaviors. For hMSCs, a similar hayflick limit was observed by these studies with reduced proliferation and altered morphology. In fact, ultra-structure of cellular organelles such as endoplasmic reticulum and matrix vesicles, was also dysregulated via replicative expansion observed under TEM. Cell cycle arrest in senescent hMSCs can be confirmed by the gradually increase of p53, p21 and p15 mRNA expressions in the results. Though DNA damage was considered as the trigger of p53/p21 signaling pathway to initiate DNA repair, independent activation of p53, p21 and p16 mRNA via metabolic regulation through AMPK and autophagy that associated with cellular homeostasis was also proposed. Loss of autophagy has been attributed to age-related stem cells functional decline. Aged muscle stem cells (muscle-SCs) and hematopoietic stem cells (HSC) acquired from rodent model revealed the impairment of autophagy in stem cell activity with chorological aging. Functionally, autophagy is protective for stem cells under internal or external stress and maintains cellular homeostasis. Muscle-SCs and HSCs lose their regenerative abilities when aged and autophagy was deficient, characterized by accumulation of autophagic vesicles, increased intracellular p62 protein levels, increased LC3II expression and ubiquitin-positive inclusions. Restoring autophagy via rapamycin and spermidine treatment also restored stem cell functions in vivo. An interesting phenomenon is the heterogeneity of aged stem cells also exhibit differential autophagy activity, which can be the similar scenario in hMSC heterogeneity. For the first time, the results demonstrated a close link between gradually loss of basal autophagy/mitophagy during culture expansion of hMSCs (FIGS. 1 and 2), which is a hallmark of loss of cellular homeostasis.

The metabolic plasticity of hMSCs has been demonstrated under artificial culture conditions. Upon removal from their in vivo niche, hMSCs start to adapt in vitro environment via utilizing both glycolysis and OXPHOS for ATP production. The pluripotency and clonal phenotype contribute to the cellular homeostasis and are maintained by this low-level of glycolytic metabolism. As metabolic shift from glycolysis towards OXPHOS was well observed in current study, breakdown of cellular homeostasis is expected due to the accumulation of damaged organelles and ROS (FIG. 3). Thus, metabolic state can act as a hallmark of replicative senescence during hMSC expansion. Proteomic and metabolomic analysis in current study revealed the metabolic alteration follows the culture expansion of hMSCs, specifically from glycolysis toward OXPHOS (FIG. 3). Moreover, genomic analysis of replicative senescent hMSCs demonstrated that genes involved in cell differentiation, apoptosis and cell death were increasingly expressed upon replicative senescence, whereas genes involved in mitosis and proliferation are down-regulated during expansion, despite of culture medium or harvest methods. Global omics provides the evidence that culture induced replicative senescence is a general property across hMSCs line despite of tissue source and culture conditions. As some controversy results exist, more investigations of the universality of hMSC replicative senescence are needed.

NAD+ has been reported to be a regulatory intermediate metabolite associated with aging in yeast and rodent, but few studies focus on in vitro expansion of human cells with senescence. The results firstly demonstrated progressive decrease of intracellular NAD+ level along with extended culture expansion of hMSCs (FIG. 4). As a redox cofactor, NAD plays a central role in energy metabolism and also acts as a substrate for enzymes involved in cellular signaling pathways, such as PARPs and Sirtuin family. Recently, studies have connected cellular NAD+ level to aging-dependent cellular functional decline in multiple organs. Replenishment of intracellular NAD+ level significantly improved the health span and thus extended the lifespan in yeast, flies, worms and mice. For stem cells, Zhang et al have reported NAD+ supplement rejuvenated senescent muscle stem cells and neural stem cell in aged mice via a regulation of mitochondrial fitness and unfolded protein response to improve metabolic activity. Interestingly, significant changes of AMPK, an energy gauge for sensing energetic homeostasis, were not observed. Thus, culture induced replicative senescence differs from muscle stem cell aging in the context of energy metabolism. As mentioned, hMSCs exhibit metabolic plasticity to adapt culture stress in order to maintain stem cells function under in vitro expansion. Metabolic shift towards OXPHOS contributed to the imbalance of NAD+/NADH redox cycle as NAD+ is rapidly converted by TCA cycle (isocitrate dehydrogenase, a-ketoglutarate dehydrogenase and malate dehydrogenase). However, since ETC-I acts as NADH dehydrogenase, senescent hMSCs with damaged mitochondria exhibited low ETC-I activity, lost the ability to maintain NAD+/NADH redox balance (FIGS. 2 and 4). Moreover, loss of autophagy/mitophagy also created a feedback loop that increased the redox imbalance (FIGS. 1 and 2). Indeed, accumulated DNA damage and continuous activation of PARP were also attributed to NAD+ depletion in senescence hMSCs.

Imai et al. proposed two major mechanisms in aging related NAD+ decline that 1) NAD+ biosynthetic pathways decline or 2) several NAD+ consumption enzymes are competing each other for the cellular NAD+ pool and culture or chronological aging induced enzymatic dysfunctions. To glance the metabolic network of NAD+ in senescent hMSCs, several key pathways for NAD+ biosynthesis and consumption were determined (FIG. 5). Surprisingly, NAMPT, which is a rate limiting step in salvage pathway for NAD+ biogenesis is highly upregulated in hMSCs with replicative senescence. This finding is contradictory with the decline of NAD+ level and can be explained by the lack of NAD+ biosynthesis substrate: NAM. Supplement of NAM has shown potential improvement of intracellular NAD+ level and recovery of mitochondria in senescent hMSCs (FIG. 6). In addition, CD38 and CD73 were highly upregulated and indicated the enhanced consumption of NAD+ in hMSCs with replicative senescence (FIG. 5). As CD38 was highly upregulated in immune cells under inflammatory environment, hMSCs cellular senescence can be connected to chorionic inflammation during culture expansion.

Based on the results, the NAD+/Sirt-1 axis can be a checkpoint for the loss of cellular function and breakdown of homeostasis. Also examined was whether human dermal fibroblasts exhibited similar mechanism during culture expansion. Surprisingly, within same population doublings (or culture period according to hMSC expansion), hFBs exhibited relative consistence in PD time and β-gal activity (FIG. 7), indicated that their cellular senescence did not increase within the expansion period. Moreover, NAD+/NADH redox balance as well as Sirt-1 (and Sirt-3) activity was well maintained. Mitochondrial function and autophagy/mitophagy were also comparable during culture expansion. These results demonstrated that hMSCs and hFBs have different sensitivity to artificial culture environment. In fact, fibroblasts were considered to share similar phenotype with MSCs, and differentiation and colony-forming ability was also observed in fibroblasts, but are highly donor and tissue source dependent. Here, was demonstrated the clear metabolic and redox difference in hFBs during culture expansion compared to hMSCs. As other studies have revealed, fibroblast can be cultured for 60-80 population doublings before entering replicative senescence, which is much more replicate times compared to hMSCs. Specifically, hFBs do not exhibit metabolic reconfiguration under nutrient-enriched environment. Instead, switching to anaerobic metabolic pathway mostly occurs in response to serum starvation. On the contrary, hMSCs is extremely sensitive to culture environment including nutrient, oxidative stress, mechanical stimuli or even gravity. hMSCs under culture stimuli exert the ability to adapt culture environment to maintain stemness and cellular function. The adaption process, however, can lead to enhanced therapeutic potentials (such as hypoxia and cytokine potentiation) or apoptosis/senescence as the marker of impropriate culture. In fact, this sensitivity provides the possibility to manipulate hMSCs with culture stimuli instead of genetic modification. For example, metabolic reconfiguration via hypoxia, 3D aggregation and cytokine pretreatment enhanced a set of hMSC properties due to metabolic plasticity. hFBs, however cannot be perturbed by the metabolic mediation since they exhibited low sensitivity to nutrient-enriched culture. Altogether, current study revealed the sensitivity of hMSCs in the context of NAD+/Sirt-1 to maintain cellular homeostasis. This study provided the strategy to delay replicative senescence and extend hMSC culture expansion with desired therapeutic quality.

### c) Materials and Methods

(1) hMSCs and hFBs Culture

Frozen hMSCs from passage 0 to 2 were acquired from Tulane Center for Gene Therapy. The MSCs were isolated from the bone marrow of healthy donors ranging in age from 19 to 49 years based on plastic adherence, negative for CD34, CD45, CD117 (all less than 2%) and positive for CD29, CD44, CD49c, CD90, CD105 and CD147 markers (all greater than 95%) and possess tri-lineage differentiation potential upon in vitro induction (Munoz 2014). hMSCs (1×106 cells/ml/vial) in freezing media contains a-MEM, 2 mM L-glutamine, 30% fetal bovine serum (FBS) and 5% dimethyl sulfoxide (DMSO) were thawed and cultured following the method. Briefly, hMSCs were expanded and maintained in complete culture media (CCM) containing a-MEM supplemented with 10% FBS (Atlanta Biologicals, Lawrenceville, Ga.) and 1% Penicillin/Streptomycin (Life Technologies, Carlsbad, Calif.) with media changed every 3 days. Cells were grown to 70%-80% confluence and then harvested by incubation with 0.25% trypsin/ethylenediaminetetraacetic acid (EDTA) (Invitrogen, Grand Island, N.Y.). Harvested cells were re-plated at a density of 1,500 cells/cm2 and sub-cultured up to Passage 15.

Primary human dermal fibroblasts (hFBs, PCS-201-012™) were purchased from American Type Culture Collection (ATCC, Manassas, Va.) and subcultured in CCM up to 15 passage. All reagents were purchased from Sigma Aldrich (St. Louis, Mo.) unless otherwise noted.

(2) Cell Number, Growth, CFU-F Assay, SA-B-Gal Activity, Comet Assay and Glucose/Lactate Measurement

Cell number was determined by Quant-iT™ PicoGreen kit (Invitrogen, Grand Island, N.Y.). Briefly, cells were harvested and lysed over-night using proteinase K (VWR, Radnor, Pa.) and stained with Picogreen to allow quantitation of cellular DNA. Fluorescence signals were read using a Fluror Count (PerkinElmer, Boston, Mass.). Population doubling time (mean PD time) was determined through culture in each passage:

$$Mean PD time = \frac{t}{{log}_{2} n}$$

where t is culture period, n is the cell number fold increase during culture time t.

Colony forming unit-fibroblast (CFU-F) were determined as following: hMSCs were harvested and re-plated at the density of 15 cells/cm2 on 60 cm2 culture dish and cultured for another 14 days in CCM. Cells were then stained with 20% crystal violet solution in methanol for 15 minutes at room temperature (RT) and washed with phosphate-buffered saline (PBS) wash, the numbers of individual colonies were counted manually.

Cellular senescence was evaluated by SA-β-Gal activity assay kit (Sigma, St. Louis, Mo.) as described in manufacture instructions.

Glucose consumption and lactate production was determined by YSI 2500 Biochemistry Select Analyzer (Yellow Spring, Ohio). Cellular DNA damage was measured by comet assay (Cell Biolabs, Inc. San Diego, Calif.), followed the manufacture instructions.

(3) Mitochondrial Morphology, Mitochondrial Mass and Membrane Potential (MMP), ROS Level Via Flow Cytometry.

For mitochondrial morphology, hMSCsc were incubated with 100 nM MitoTracker Red CMXRos (Molecular Probe, Eugene, Oreg.) in complete culture medium at 37° C. for 30 minutes. After washing, cells were then incubated with 3.7% formaldehyde at 37° C. for 15 minutes. Imaging was performed using Olympus IX70 microscope. Mitochondrial shape factors, including circularity (4×π area)/perimeter), aspect ratio (largest diameter/smallest diameter), and nucleus to cytoplasm ratio were quantified using Image J (NIH software).

For MMP measurement, trypsinized MSCs were washed by centrifugation in warm Hank's Balanced Salt Solution (HBSS). Cell suspension was incubated with tetramethylrhodamine, methyl ester (TMRM) (Molecular Probe, Eugene, Oreg.) at 37° C., washed with HBSS, and analyzed by flow cytometry (BD Biosciences, San Jose, Calif.).

For ROS measurement, aliquots of cell suspension were incubated with 25 μM carboxy-H2DCFDA (Molecular Probe, Eugene, Oreg.) at 37° C. for 30 minutes. The intracellular ROS levels were determined using flow cytometry (BD Biosciences, San Jose, Calif.). For mitochondrial ROS measurement, aliquots of cell suspension were incubated with 5 μM MitoSOX Red (Molecular Probe, Eugene, Oreg.) at 37° C. for 10 minutes and analyzed using flow cytometry (BD Biosciences, San Jose, Calif.).

(4) Immunocytochemistry, Cell Cycle, Autophagy and Mitophagy Measurement

Cells were dissociated from monolayer MSCs or MSC aggregates by incubation with 0.25% trypsin-EDTA solution for 10-15 min at 37° C. Suspended MSCs were washed in phosphate-buffered saline (PBS), and fixed at 4% paraformaldehyde (PFA) at room temperature for 15 minutes. Cells were then permeabilized in 0.2% triton X-100 PBS for 10 min at room temperature (RT). Non-specific binding sites were blocked in PBS with 1% bovine serum albumin, 10% goat serum, 4% nonfat dry milk for 15 min at RT. After washing with PBS, cells were incubated with specific primary antibodies for human Sirt-1, Sirt-3, NAMPT, CD38, CD73 (Santa Cruz Biotechnology, Dallas, Tex.) at RT for 2 h, following with 1 h incubation with FITC conjugated secondary antibody (Molecular Probe, Eugene, Oreg.) at RT. Labeled samples were washed in PBS followed by flow cytometry analysis with isotype control run in parallel.

For autophagy measurement, aliquots of cell suspension were incubated with 20 μM Cyto-ID Green (Enzo Life Sciences, Farmingdale, N.Y.), a fluorescent dye that selectively labels accumulated autophagic vacuoles, at 37° C. for 30 minutes, and analyzed by flow cytometry (BD Biosciences, San Jose, Calif.). For mitophagy measurement,

(5) Intracellular ATP Content and Mitochondrial Complex I Activity

hMSCs were centrifuged, re-suspended in DI water, and heated immediately in a boiling water bath for 15 minutes. The mixture was centrifuged and ATP containing supernatant was collected. Upon measurement, 10 μL of ATP solution was mixed with 100 μL of the luciferin-luciferase reagent, and the bioluminescent signal was measured using an Orion Microplate Luminometer (Bad Wildbad, Deutschland) after 15 min incubation at room temperature. Activity of mitochondrial complex I was determined using the Complex I Enzyme Activity Microplate Assay Kit (Abcam, Cambridge, Mass.) according to the kit instructions.

(6) Real-time Reverse Transcriptase-Polymerase Chain Reaction (RT-PCR)

Total RNA was isolated using the RNeasy Plus kit (Qiagen) following vendor's instructions. Reverse transcription was carried out using 2 μg of total RNA, anchored oligo-dT primers (Operon) and Superscript III (Invitrogen). Primers for specific target genes were designed using the software Oligo Explorer 1.2 (Genelink). β-actin was used as an endogenous control for normalization. Real-time reverse transcriptase-polymerase chain reaction (RT-PCR) reactions were performed on an ABI7500 instrument (Applied Biosystems), using SYBR Green PCR Master Mix. The amplification reactions were performed and the quality and primer specificity were verified. hMSCs without any treatment were used as control group to determine relative fold-increase. Fold variation in gene expressions were quantified using the comparative Ct method: 2−(CtTreatment−CtControl), which is based on the comparison of the expression of the target gene (normalized to β-actin).

(7) Proteomic Analysis

Cells were harvested when reached 80% confluency from 2D planer culture. hMSC pellets were washed with cold PBS for three times for complete removal of medium. Cell pellets were resuspended in protein extracting buffer supplied with protease inhibitor. 2-min ultra-sonication was used to extract proteins of cells on ice. Protein concentration was determined by Bradford assay (Bio-Rad, Hercules, Calif.). Extracted proteins were digested by modified Filter Aided Sample Prep (FASP) method. Briefly, 100 μg protein was vacuum-dried and resuspended in 8 M urea solution to a final volume of 200 μL, then 10 mM dithiothreitol (DTT) and 50 mM iodoacetamide (IAA) were added for reduction and alkylation respectively. Samples were transferred to a 10 kDa filter and centrifuged with 14000 g for 30 minutes to eliminate solvent. Two times of wash was performed with 200 μL 8 M urea and 200 μL ammonium bicarbonate and the extracts were centrifuged at 14000 g for 30 minutes to remove solvent. Then 2 μg trypsin was added for digestion under 37° C. overnight. After that, peptides were collected and vacuum-dried. An externally calibrated Thermo Q Exactive HF (high-resolution electrospray tandem mass spectrometer, MS, Thermo Scientific) was used in conjunction with Dionex UltiMate3000 RSLCnano System. 1 mg microgram of peptides resuspended in 0.1% formic acid was injected into a 50 μL loop and loaded onto the trap column (Thermo μ-Precolumn 5 mm, with nanoViper tubing 30 μm i.d.×10 cm). The flow rate was set to 300 nL/min for separation on the analytical column (Acclaim pepmap RSLC 75 μM×15 cm nanoviper). Mobile phase A was composed of 99.9% H₂O (EMD Omni Solvent) with 0.1% formic acid and mobile phase B was composed of 99.9% acetonitrile with 0.1% formic acid. A 120 mins stepped gradient from 3% to 45% phase B was performed. The LC eluent was directly nano-sprayed into Q Exactive HF MS. During the chromatographic separation, the Q Exactive HF was operated in a data-dependent mode and under direct control of the Thermo Excalibur 3.1.66 (Thermo Scientific). The MS data were acquired using the following parameters: 20 data-dependent collisional-induced-dissociation (CID) MS/MS scans per full scan (350 to 1700 m/z). The spray voltage for Thermo Scientific™ LTQ was 2.0 kV and the capillary temperature was set at 200° C. A survey full scan (m/z=350-1700) and the five most intense ions were selected for a zoom scan to determine the charge state, after which MS/MS was triggered in Pulsed-Q Dissociation mode (PQD) with minimum signal required (1000), isolation width 2.0, normalized collision energy 27.0. All measurements were performed at room temperature. Raw files were analyzed by Maxquant 1.6 followed protein identification and relative comparison in Scaffold 4.4. Gene ontology (GO) annotation was carried out by WebGestalt while canonical pathway, diseases and functions analysis was performed by Ingenuity Pathway Analysis (IPA, Qiagen).

(8) Statistics/Data Analysis

Unless otherwise noted, all experiments were performed at least in triplicate (n=3), and representative data are reported. Experimental results are expressed as means±standard deviation (SD) of the samples. Statistical comparisons were performed by one-way ANOVA and Tukey's post hoc test for multiple comparisons, and significance was accepted at p<0.05.

### 2. Example 2: Metabolic Regulation of Cellular Homeostasis in Human Mesenchymal Stem Cells During In Vitro Culture Expansion

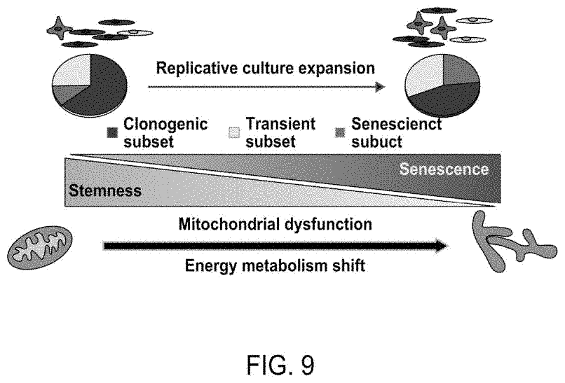

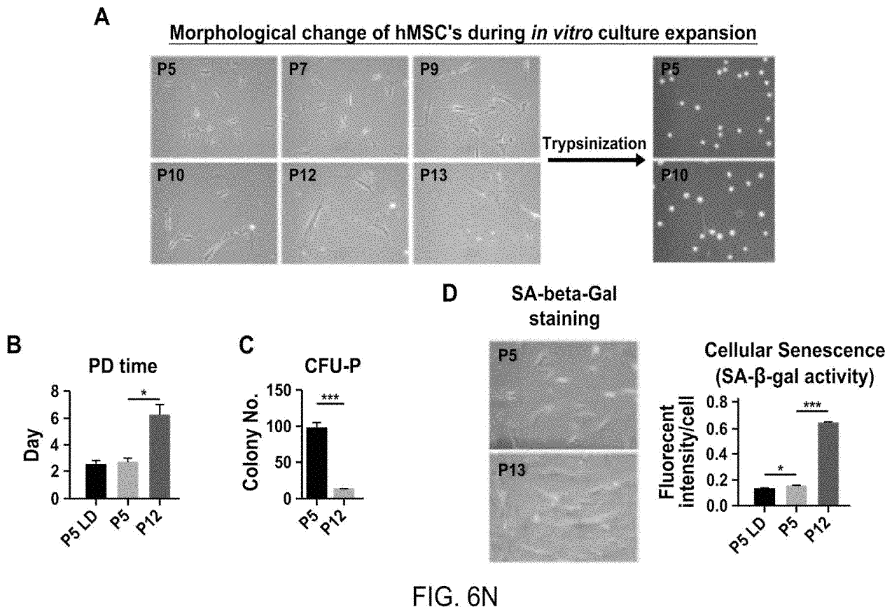

The objective of this study is to investigate long-term culture-induced changes of hMSCs which leads to cellular senescence and metabolic alteration, resulting in reduced stem cell properties; and to understand the key regulatory role of NAD+/NADH redox cycle and mitochondria in maintaining energy, metabolic profile, and preserving cellular homeostasis during hMSCs in vitro expansion. As shown in FIG. 9, hMSCs are cultured in vitro for expansion purpose, with the increase of senescent population and decline of stem cell properties. Cellular compartments are dysfunctional during replicative expansion, leading to imbalance of energy and cellular homeostasis.

a) Methods

hMSCs at passage 2 were expanded and cultured in complete culture medium(CCM). Cells were grown to 70-80% confluence and then harvested by incubation with 0.25% trypsin/EDTA. Harvested cells were re-plated at a density of 2,000 cells/cm² and sub-cultured up to Passage 12 in a standard CO₂ incubator. hMSC sat Passage 5 (P5) and passage 12 (P12) were used for experiments. Stem cell properties including senescence, stem cell gene, cell cycle, proliferation rate were determined. Gas chromatography-mass spectrometry (GC-MS) and seahorse flux analyzer were used for characterization of energy metabolism phenotype. Mitochondrial functions were assessed by mitochondrial mass, membrane potential, electron transport complex activity and biogenesis. NAD+/NADH and Sirt-1 and -3 level was also measured in P5 and P12 hMSCs. NAD+ precursor nicotinamide (NAM) was used in CCM to adjust NAD+ level intracellularly.

b) Results

(1) Decline of Stem Cell Properties of hMSCs Due to Replicative Culture Expansion In Vitro

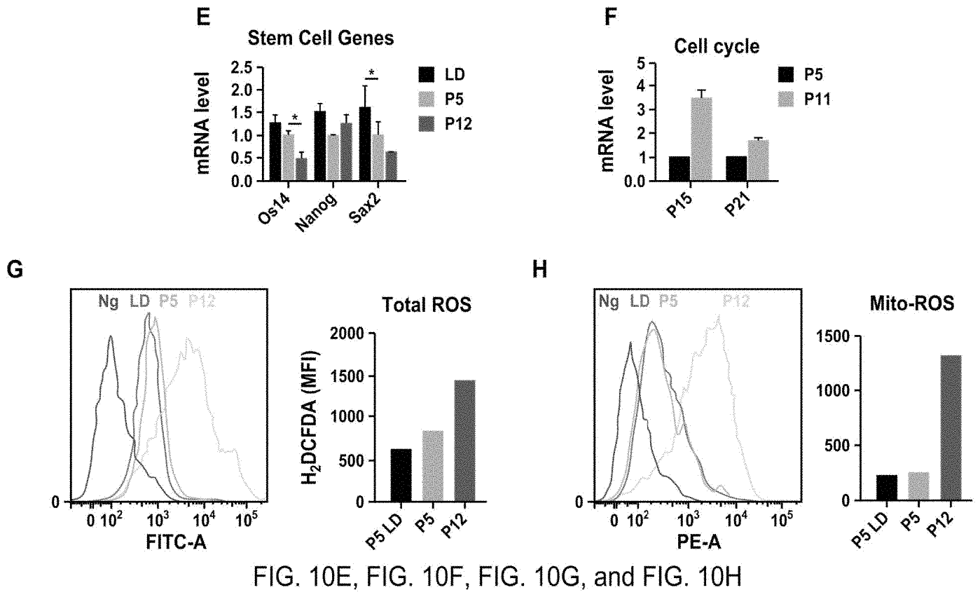

FIGS. 10A-10H show culture induced hMSC senescence and function decline. FIG. 10A shows hMSC morphology and size alteration during in vitro culture expansion; (10B) population doubling time, (10C) colony forming ability and (10D) cellular senescence of hMSCs at different passages; (10E) Stem cell gene and (10F) cell cycle gene of hMSCs at different passage; (10G) Total reactive oxygen species (ROS) and (10H) mitochondrial ROS of hMSCs at different passages.

(2) Metabolic Reconfiguration of hMSCs During Replicative Culture Expansion In Vitro

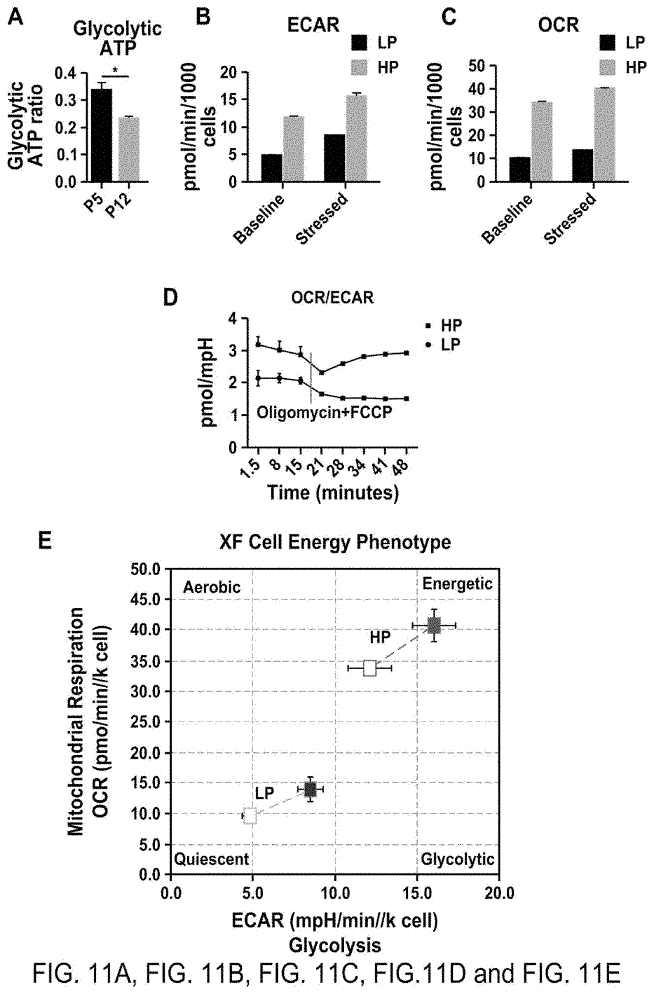

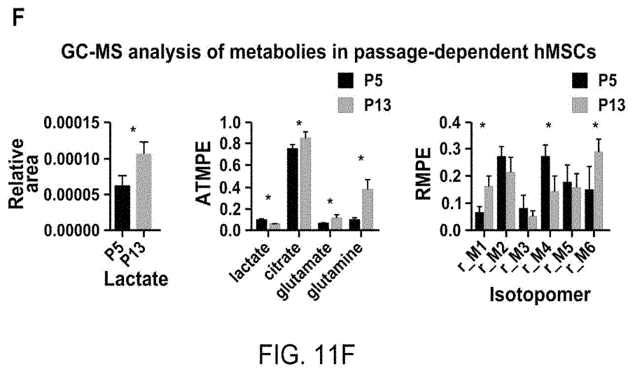

FIGS. 11A-11F show metabolic reconfiguration in replicative cultured hMSCs. FIG. 11A shows Glycolytic ATP of passage-dependent hMSCs; (11B) Extracellular acidification rate(ECAR), (11C) oxygen consumption rate (OCR), (11D) OCR/ECAR and (11E) metabolic potential of hMSCs at different passage; (11F) GC-MS analysis of metabolites involved in glycolysis and TCA cycle, shown as absolute molar percent enrichments (ATMPE) and relative molar percent enrichments (RMPE).

(3) Mitochondrial Dysfunction of hMSCs During In Vitro Culture Expansion

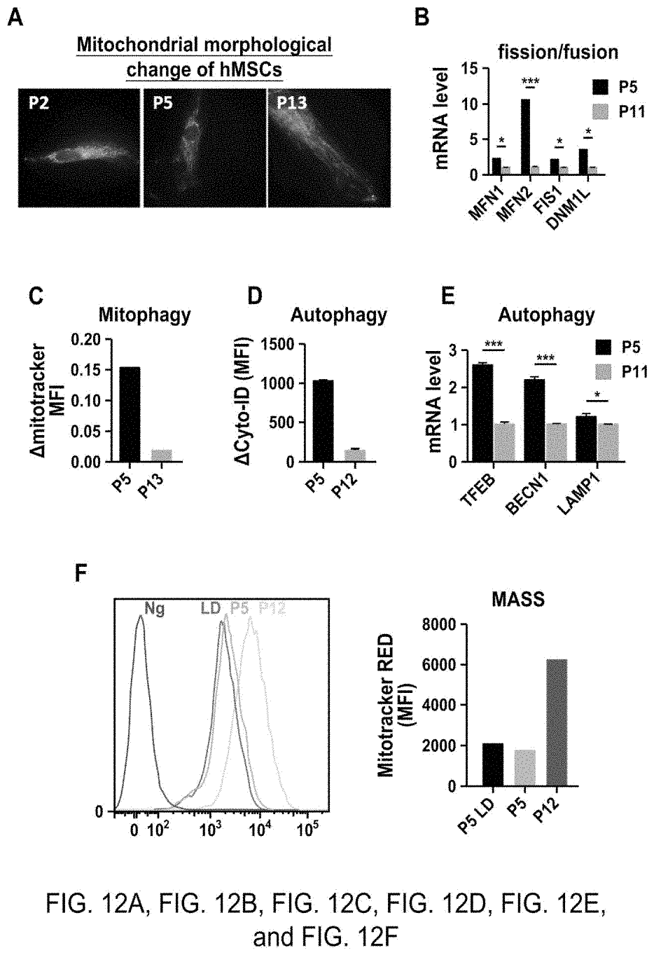

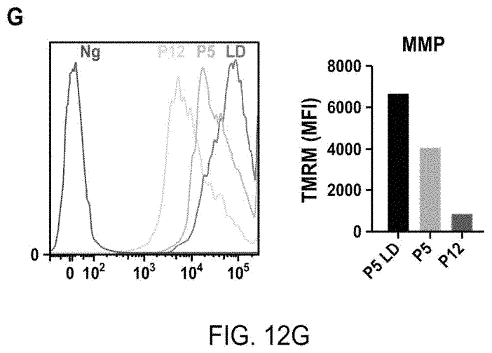

FIGS. 12A-12G show mitochondrial function decline in replicative expanded hMSCs. FIG. 12A shows mitochondrial morphology change, (12B) mitochondrial fusion and fission gene, (12C) mitophagy and (12D) autophagy, (12E) mitophagy and autophagy gene in hMSCs at different passages; (12F) mitochondrial mass and (12G) membrane potential hMSCs from different passages.

(4) NAD+ Decline in Culture-Induced Senescent hMSCs and NAD+/Sirtuin Axis Regulates hMSC Cellular Homeostasis

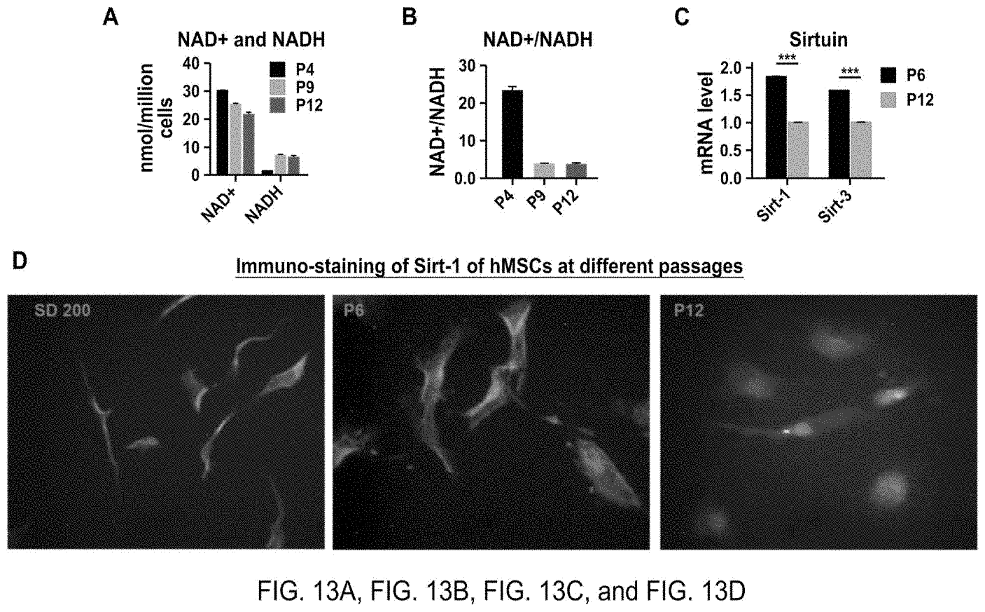

FIGS. 13A-13D show Immuno-staining of Sirt-1 of hMSCs at different passages NAD+/NADH redox cycle was imbalanced in long-term cultured hMSCs. FIG. 13A shows intracellular NAD+ and NADH level and (13B) NAD+/NADH ratio in hMSCs at different passages; (13C) Gene expression of Sirt-1 and Sirt-3 in hMSCs at different passages; (13D) Sirt-1 expression in hMSCs via ICC staining.

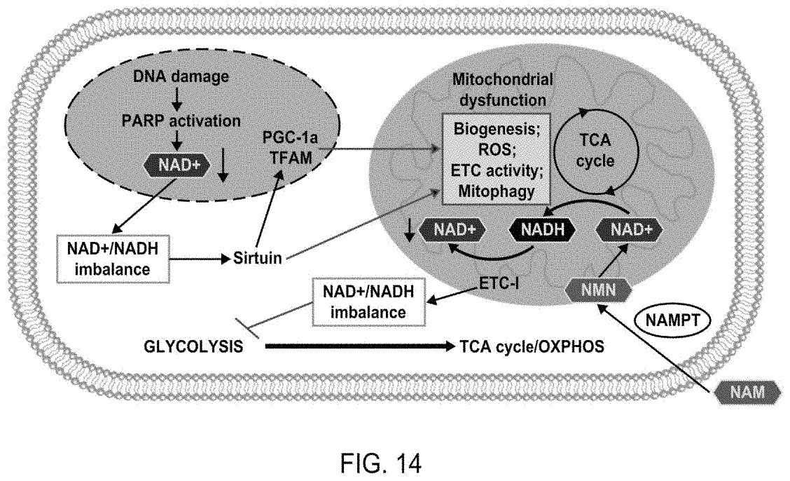

FIG. 14 shows the mechanism of how NAD+/NADH and mitochondria regulate hMSCs cellular homeostasis during in vitro culture expansion. Culture expansion of hMSCs results in accumulation of DNA damage, which further activates PARP signal and causes the intracellular NAD+ decrease. Imbalanced NAD+/NADH level causes NAD+ dependent Sirtuin inactivation, which down-regulates several pathways, including mitochondrial biogenesis, anti-oxydant protection, and immunomodulation. Dysfunction of mitochondria further accumulates NADH and consumes NAD+ to maintain cellular function, leading energy metabolism shift from glycolysis towards OXPHOS. Maintaining intracellular NAD+ pool size via supplement of NAD+ precursors could enhance hMSCs resistance to senescence during replicative expansion.

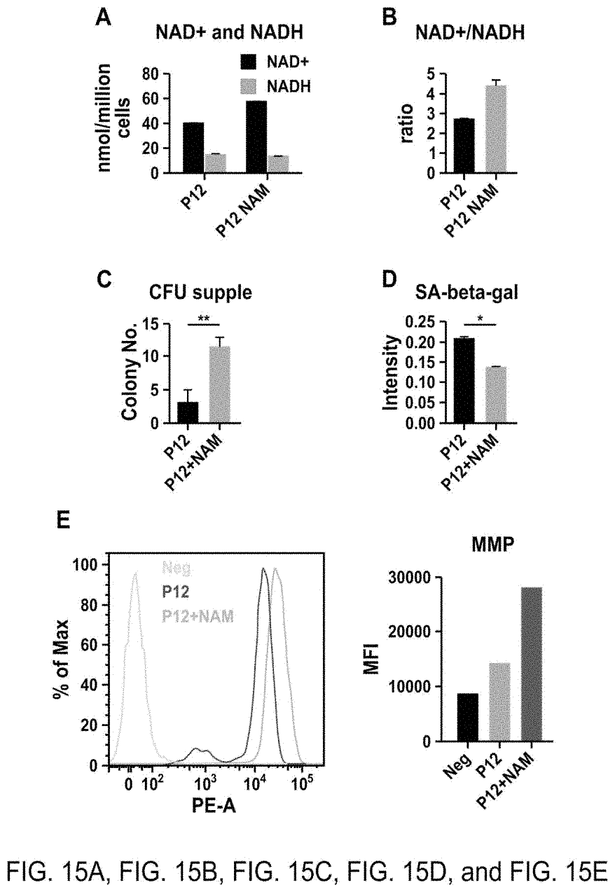

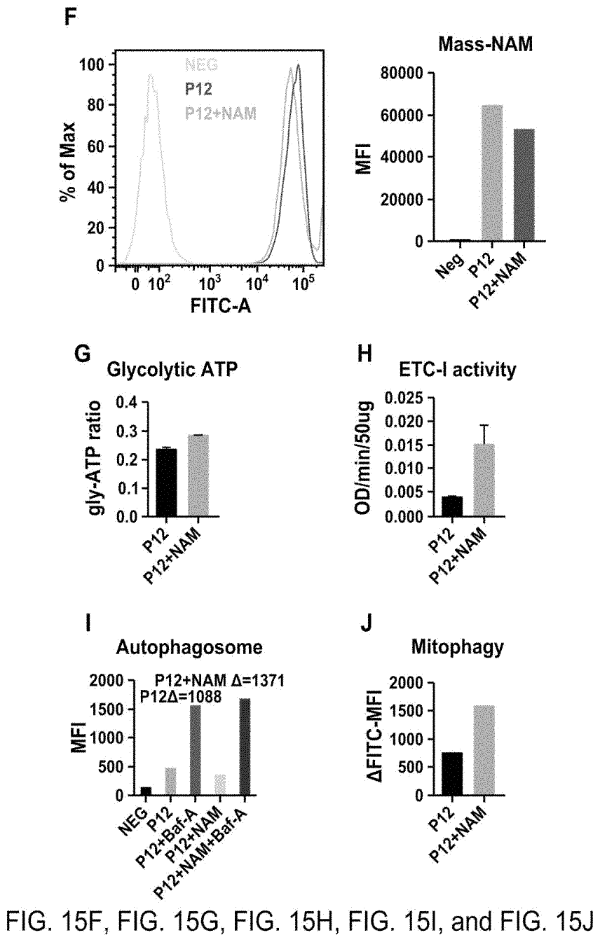

FIGS. 15A-15J show the rejuvenation of hMSC cellular homeostasis via changing intracellular NAD+ level. FIG. 15A shows NAD+ and NADH level and (15B) NAD+NADH ratio after supplement of NAD+ precursor in culture induced senescent hMSCs; (15C) CFU ability and (15D) cellular senescence of high passage hMSCs with supplement; (15E) mitochondrial membrane potential and (15F) mass in high passage hMSCs with supplement; (15G) Glycolysis, (15H) mitochondrial ETC activity, (15D autophagy and (15J) mitophagy were also enhanced by NAD+ precursor supplement in culture induced senescent hMSCs.

### 3. Example 3: NAD+ Precursors can Prevent Culture Induced Senescence and Rescue Cellular Functions

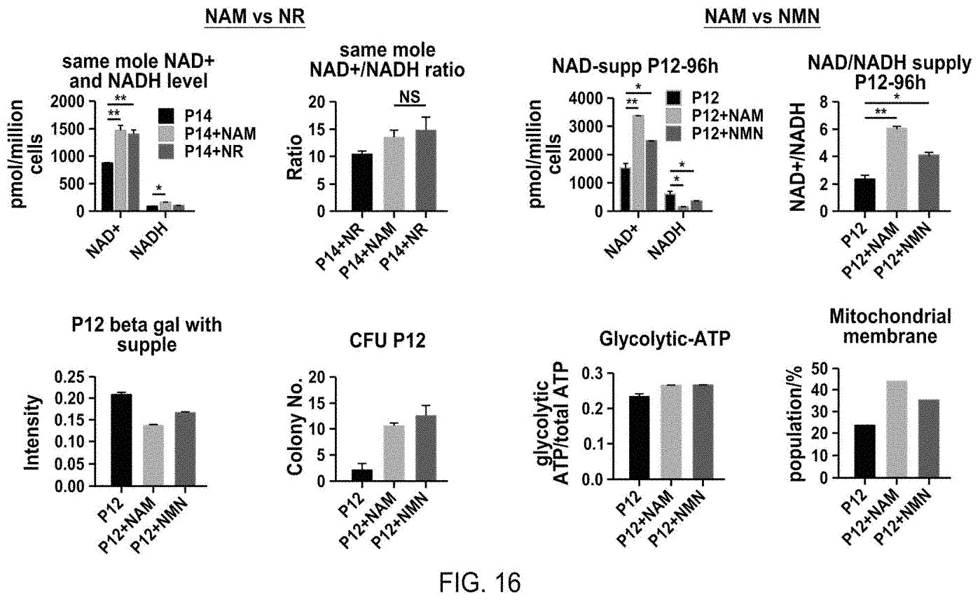

FIG. 16 shows a comparison of the effects of various NAD+ precursors on stem cells in culture. In detail, NAM and NR have no significant difference in boosting intracellular NAD+ level and RAD+/NADH ratio. However, NAM is better in boosting intracellular NAD+ level compared to NMN and this has been tested throughout the rejuvenation of cellular senescence, glycolytic activity and mitochondrial fitness.

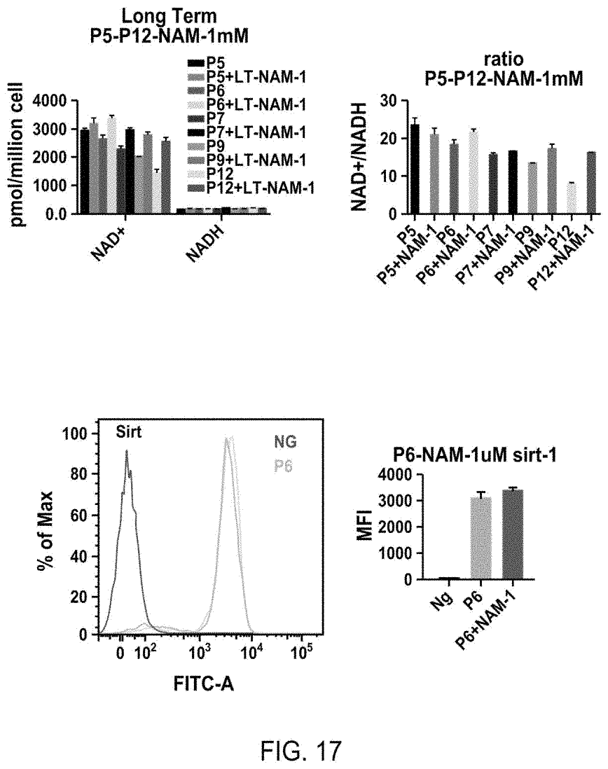

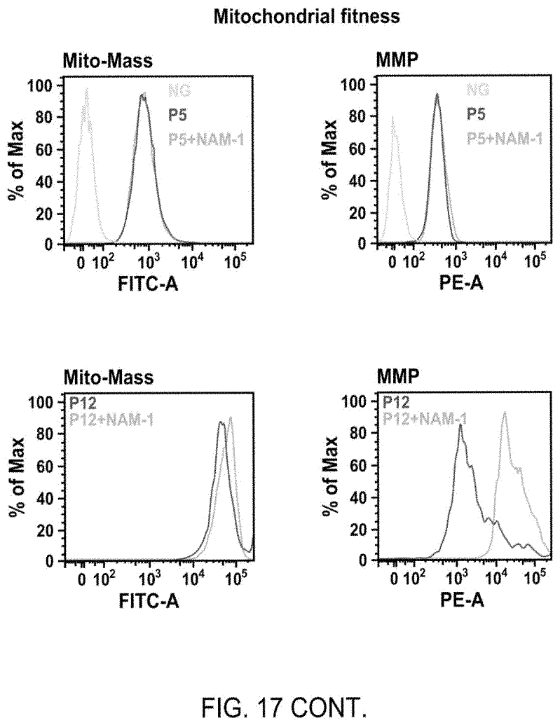

FIG. 17 shows that a continuous supplement of NAD+ precursor NAM (1 mM) maintains hMSC cellular NAD+ level and Sirt activity, as well as mitochondrial fitness.

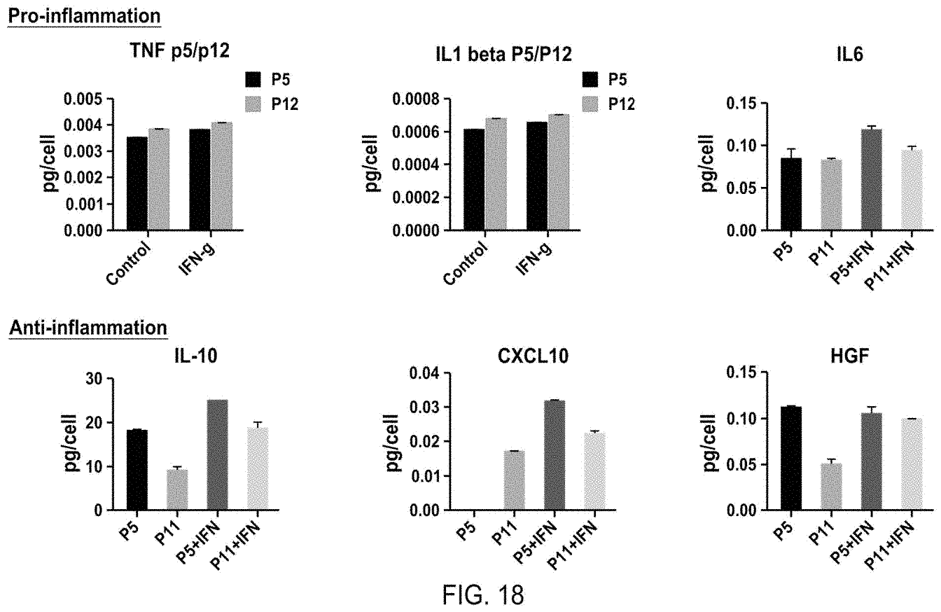

FIG. 18 shows the decreased immunomodulatory potentials of senescent hMSCs with significantly higher pro-inflammation.).

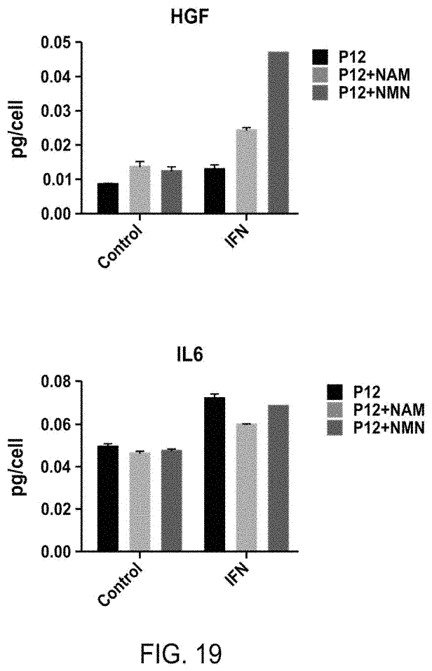

FIG. 19 shows that NAD+ supplement restores hMSC immunomodulation with potent anti-inflammation

### D. REFERENCES

A. C. Tsai, Y. Liu, X. Yuan, T. Ma, Compaction, fusion, and functional activation of three-dimensional human mesenchymal stem cell aggregate, Tissue Eng Part A 21(9-10) (2015) 1705-19.
A. F. Santidrian, A. Matsuno-Yagi, M. Ritland, B. B. Seo, S. E. LeBoeuf, L. J. Gay, T. Yagi, B. Felding-Habermann, Mitochondrial complex I activity and NAD+/NADH balance regulate breast cancer progression, J Clin Invest 123(3) (2013) 1068-81.
A. P. Gomes, N. L. Price, A. J. Ling, J. J. Moslehi, M. K. Montgomery, L. Rajman, J. P. White, J. S. Teodoro, C. D. Wrann, B. P. Hubbard, E. M. Mercken, C. M. Palmeira, R. de Cabo, A. P. Rolo, N. Turner, E. L. Bell, D. A. Sinclair, Declining NAD(+) induces a pseudohypoxic state disrupting nuclear-mitochondrial communication during aging, Cell 155(7) (2013) 1624-38.
B. A. Scruggs, J. A. Semon, X. Zhang, S. Zhang, A. C. Bowles, A. C. Pandey, K. M. Imhof, A. V. Kalueff, J. M. Gimble, B. A. Bunnell, Age of the donor reduces the ability of human adipose-derived stem cells to alleviate symptoms in the experimental autoimmune encephalomyelitis mouse model, Stem Cells Transl Med 2(10) (2013) 797-807.
E. Alt, Y. Yan, S. Gehmert, Y. H. Song, A. Altman, S. Gehmert, D. Vykoukal, X. Bai, Fibroblasts share mesenchymal phenotypes with stem cells, but lack their differentiation and colony-forming potential, Biol Cell 103(4) (2011) 197-208.
E. F. Fang, H. Kassahun, D. L. Croteau, M. Scheibye-Knudsen, K. Marosi, H. Lu, R. A. Shamanna, S. Kalyanasundaram, R. C. Bollineni, M. A. Wilson, W. B. Iser, B. N. Wollman, M. Morevati, J. Li, J. S. Kerr, Q. Lu, T. B. Waltz, J. Tian, D. A. Sinclair, M. P. Mattson, H. Nilsen, V. A. Bohr, NAD(+) Replenishment Improves Lifespan and Healthspan in Ataxia Telangiectasia Models via Mitophagy and DNA Repair, Cell Metab 24(4) (2016) 566-581.
E. N. Chini, C. C. S. Chini, J. M. Espindola Netto, G. C. de Oliveira, W. van Schooten, The Pharmacology of CD38/NADase: An Emerging Target in Cancer and Diseases of Aging, Trends Pharmacol Sci 39(4) (2018) 424-436.
F. Sabatini, L. Petecchia, M. Tavian, V. Jodon de Villeroche, G. A. Rossi, D. Brouty-Boye, Human bronchial fibroblasts exhibit a mesenchymal stem cell phenotype and multilineage differentiating potentialities, Lab Invest 85(8) (2005) 962-71.
G. Siegel, T. Kluba, U. Hermanutz-Klein, K. Bieback, H. Northoff, R. Schafer, Phenotype, donor age and gender affect function of human bone marrow-derived mesenchymal stromal cells, BMC Med 11 (2013) 146.
H. Massudi, R. Grant, N. Braidy, J. Guest, B. Farnsworth, G. J. Guillemin, Age-associated changes in oxidative stress and NAD+ metabolism in human tissue, PloS one 7(7) (2012) e42357.
H. Zhang, D. Ryu, Y. Wu, K. Gariani, X. Wang, P. Luan, D. D'Amico, E. R. Ropelle, M. P. Lutolf, R. Aebersold, K. Schoonjans, K. J. Menzies, J. Auwerx, NAD(+) repletion improves mitochondrial and stem cell function and enhances life span in mice, Science 352(6292) (2016) 1436-43.
H. E. Gruber, S. Somayaji, F. Riley, G. L. Hoelscher, H. J. Norton, J. Ingram, E. N. Hanley, Jr., Human adipose-derived mesenchymal stem cells: serial passaging, doubling time and cell senescence, Biotech Histochem 87(4) (2012) 303-11.
I. Horikawa, K. Fujita, L. M. Jenkins, Y. Hiyoshi, A. M. Mondal, B. Vojtesek, D. P. Lane, E. Appella, C. C. Harris, Autophagic degradation of the inhibitory p53 isoform Delta133p53alpha as a regulatory mechanism for p53-mediated senescence, Nat Commun 5 (2014) 4706.
J. Camacho-Pereira, M. G. Tarrago, C. C. S. Chini, V. Nin, C. Escande, G. M. Warner, A. S. Puranik, R. A. Schoon, J. M. Reid, A. Galina, E. N. Chini, CD38 Dictates Age-Related NAD Decline and Mitochondrial Dysfunction through an SIRT3-Dependent Mechanism, Cell Metab 23(6) (2016) 1127-1139.
J. Campisi, Replicative senescence: an old lives' tale?, Cell 84(4) (1996) 497-500.
J. Yoshino, K. F. Mills, M. J. Yoon, S. Imai, Nicotinamide mononucleotide, a key NAD(+) intermediate, treats the pathophysiology of diet- and age-induced diabetes in mice, Cell Metab 14(4) (2011) 528-36.
J. C. Estrada, Y. Tones, A. Benguria, A. Dopazo, E. Roche, L. Carrera-Quintanar, R. A. Perez, J. A. Enriquez, R. Tones, J. C. Ramirez, E. Samper, A. Bernad, Human mesenchymal stem cell-replicative senescence and oxidative stress are closely linked to aneuploidy, Cell Death Dis 4 (2013) e691.
J. C. Medeiros Tavares Marques, D. A. Cornelio, V. Nogueira Silbiger, A. Ducati Luchessi, S. de Souza, S. R. Batistuzzo de Medeiros, Identification of new genes associated to senescent and tumorigenic phenotypes in mesenchymal stem cells, Sci Rep 7(1) (2017) 17837.
J. C. Sepulveda, M. Tome, M. E. Fernandez, M. Delgado, J. Campisi, A. Bernad, M. A. Gonzalez, Cell senescence abrogates the therapeutic potential of human mesenchymal stem cells in the lethal endotoxemia model, Stem Cells 32(7) (2014) 1865-77.
K. Schallmoser, C. Bartmann, E. Rohde, S. Bork, C. Guelly, A. C. Obenauf, A. Reinisch, P. Horn, A. D. Ho, D. Strunk, W. Wagner, Replicative senescence-associated gene expression changes in mesenchymal stromal cells are similar under different culture conditions, Haematologica 95(6) (2010) 867-74.
L. Danisovic, L. Oravcova, L. Krajciova, Z. Varchulova Novakova, M. Bohac, I. Varga, J. Vojtassak, Effect of long-term culture on the biological and morphological characteristics of human adipose tissue-derived stem Cells, J Physiol Pharmacol 68(1) (2017) 149-158.
L. Garcia-Prat, M. Martinez-Vicente, E. Perdiguero, L. Ortet, J. Rodriguez-Ubreva, E. Rebollo, V. Ruiz-Bonilla, S. Gutarra, E. Ballestar, A. L. Serrano, M. Sandri, P. Munoz-Canoves, Autophagy maintains sternness by preventing senescence, Nature 529(7584) (2016) 37-42.
L. Guarente, Mitochondria—a nexus for aging, calorie restriction, and sirtuins?, Cell 132(2) (2008) 171-6.
L. Mouchiroud, R. H. Houtkooper, N. Moullan, E. Katsyuba, D. Ryu, C. Canto, A. Mottis, Y. S. Jo, M. Viswanathan, K. Schoonjans, L. Guarente, J. Auwerx, The NAD(+)/Sirtuin Pathway Modulates Longevity through Activation of Mitochondrial UPR and FOXO Signaling, Cell 154(2) (2013) 430-41.
L. M. Perez, A. Bernal, B. de Lucas, N. San Martin, A. Mastrangelo, A. Garcia, C. Barbas, B. G. Galvez, Altered metabolic and sternness capacity of adipose tissue-derived stem cells from obese mouse and human, PloS one 10(4) (2015) e0123397.
M. Golpour, H. Akhavan Niaki, H. R. Khorasani, A. Hajian, R. Mehrasa, A. Mostafazadeh, Human fibroblast switches to anaerobic metabolic pathway in response to serum starvation: a mimic of warburg effect, Int J Mol Cell Med 3(2) (2014) 74-80.
M. Murata, X. Kong, E. Moncada, Y. Chen, P. H. Wang, M. W. Berns, K. Yokomori, M. Digman, NAD consumption by PARP1 in response to DNA damage triggers metabolic shift critical for damaged cell survival, bioRxiv (2018) 375212.
M. Revuelta, A. Matheu, Autophagy in stem cell aging, Aging Cell 16(5) (2017) 912-915.
M. Soundararajan, S. Kannan, Fibroblasts and mesenchymal stem cells: Two sides of the same coin?, J Cell Physiol 233(12) (2018) 9099-9109.
M. S. Bonkowski, D. A. Sinclair, Slowing ageing by design: the rise of NAD(+) and sirtuin-activating compounds, Nat Rev Mol Cell Biol 17(11) (2016) 679-690.
M. U. Awan, Y. Deng, Role of autophagy and its significance in cellular homeostasis, Appl Microbiol Biotechnol 98(12) (2014) 5319-28.
N. Munoz, J. Kim, Y. Liu, T. M. Logan, T. Ma, Gas chromatography-mass spectrometry analysis of human mesenchymal stem cell metabolism during proliferation and osteogenic differentiation under different oxygen tensions, J Biotechnol 169 (2014) 95-102.
P. Bai, C. Canto, H. Oudart, A. Brunyanszki, Y. Cen, C. Thomas, H. Yamamoto, A. Huber, B. Kiss, R. H. Houtkooper, K. Schoonjans, V. Schreiber, A. A. Sauve, J. Menissier-de Murcia, J. Auwerx, PARP-1 inhibition increases mitochondrial metabolism through SIRT1 activation, Cell Metab 13(4) (2011) 461-468.
P. Belenky, F. G. Racette, K. L. Bogan, J. M. McClure, J. S. Smith, C. Brenner, Nicotinamide riboside promotes Sir2 silencing and extends lifespan via Nrk and Urhl/Pnpl/Meul pathways to NAD+, Cell 129(3) (2007) 473-84.
P. K. Wu, J. Y. Wang, C. F. Chen, K. Y. Chao, M. C. Chang, W. M. Chen, S. C. Hung, Early Passage Mesenchymal Stem Cells Display Decreased Radiosensitivity and Increased DNA Repair Activity, Stem Cells Transl Med 6(6) (2017) 1504-1514.
R. Meng, H. Y. Xu, S. M. Di, D. Y. Shi, A. R. Qian, J. F. Wang, P. Shang, Human mesenchymal stem cells are sensitive to abnormal gravity and exhibit classic apoptotic features, Acta Biochim Biophys Sin (Shanghai) 43(2) (2011) 133-42.
R. A. Denu, S. Nemcek, D. D. Bloom, A. D. Goodrich, J. Kim, D. F. Mosher, P. Hematti, Fibroblasts and Mesenchymal Stromal/Stem Cells Are Phenotypically Indistinguishable, Acta Haematol 136(2) (2016) 85-97.
S. Imai, L. Guarente, NAD+ and sirtuins in aging and disease, Trends in cell biology 24(8) (2014) 464-71.
S. Zha, Z. Li, Q. Cao, F. Wang, F. Liu, PARP1 inhibitor (PJ34) improves the function of aging-induced endothelial progenitor cells by preserving intracellular NAD(+) levels and increasing SIRT1 activity, Stem Cell Res Ther 9(1) (2018) 224.
T. Yang, N.Y. Chan, A. A. Sauve, Syntheses of nicotinamide riboside and derivatives: effective agents for increasing nicotinamide adenine dinucleotide concentrations in mammalian cells, J Med Chem 50(26) (2007) 6458-61.
T. T. Ho, M. R. Warr, E. R. Adelman, O. M. Lansinger, J. Flach, E. V. Verovskaya, M. E. Figueroa, E. Passegue, Autophagy maintains the metabolism and function of young and old stem cells, Nature 543(7644) (2017) 205-210.
U. Herbig, W. A. Jobling, B. P. C. Chen, D. J. Chen, J. M. Sedivy, Telomere shortening triggers senescence of human cells through a pathway involving ATM, p53, and p21(CIP1), but not p16(INK4a), Mol Cell 14(4) (2004) 501-513.
V. Balan, G. S. Miller, L. Kaplun, K. Balan, Z. Z. Chong, F. Li, A. Kaplun, M. F. VanBerkum, R. Arking, D. C. Freeman, K. Maiese, G. Tzivion, Life span extension and neuronal cell protection by Drosophila nicotinamidase, J Biol Chem 283(41) (2008) 27810-9.
W. Wagner, A. D. Ho, M. Zenke, Different facets of aging in human mesenchymal stem cells, Tissue engineering. Part B, Reviews 16(4) (2010) 445-53.
W. Wagner, P. Horn, M. Castoldi, A. Diehlmann, S. Bork, R. Saffrich, V. Benes, J. Blake, S. Pfister, V. Eckstein, A. D. Ho, Replicative senescence of mesenchymal stem cells: a continuous and organized process, PloS one 3(5) (2008) e2213.
W. Wang, X. Yang, I. Lopez de Silanes, D. Carling, M. Gorospe, Increased AMP:ATP ratio and AMP-activated protein kinase activity during cellular senescence linked to reduced HuR function, J Biol Chem 278(29) (2003) 27016-23.
X. Meng, M. Xue, P. Xu, F. Hu, B. Sun, Z. Xiao, MicroRNA profiling analysis revealed different cellular senescence mechanisms in human mesenchymal stem cells derived from different origin, Genomics 109(3-4) (2017) 147-157.
X. Yuan, A. C. Tsai, I. Farrance, J. Rowley, T. Ma, Aggregation of Culture Expanded Human Mesenchymal Stem Cells in Microcarrier-based Bioreactor, Biochem Eng J 131 (2018) 39-46.
X. H. Zhu, M. Lu, B. Y. Lee, K. Ugurbil, W. Chen, In vivo NAD assay reveals the intracellular NAD contents and redox state in healthy human brain and their age dependences, Proc Natl Acad Sci USA 112(9) (2015) 2876-81.
Y. Liu, A.-C. Tsai, X. Yuan, T. Ma, The effect of three-dimensional aggregates on the biology of mesenchymal stromal cells, The Biology and Therapeutic Application of Mesenchymal Cells, John Wiley & Sons, Inc.2016, pp. 75-90.
Y. Liu, A.-C. Tsai, X. Yuan, Y. Li, T. Ma, Chapter 18-Hypoxia Regulation of Stem Cell: Mechanisms, Biological Properties, and Applications, in: A. Vishwakarma, J. M. Karp (Eds.), Biology and Engineering of Stem Cell Niches, Academic Press, Boston, 2017, pp. 273-291.
Y. Liu, N. Munoz, A. C. Tsai, T. M. Logan, T. Ma, Metabolic Reconfiguration Supports Reacquisition of Primitive Phenotype in Human Mesenchymal Stem Cell Aggregates, Stem Cells 35(2) (2017) 398-410.
Y. Liu, N. Munoz, B. A. Bunnell, T. M. Logan, T. Ma, Density-Dependent Metabolic Heterogeneity in Human Mesenchymal Stem Cells, Stem Cells 33(11) (2015) 3368-81.
Y. Liu, X. Yuan, N. Munoz, T. M. Logan, T. Ma, Commitment to Aerobic Glycolysis Sustains Immunosuppression of Human Mesenchymal Stem Cells, Stem Cells Transl Med 8(1) (2019) 93-106.
Y. Yuan, V. F. Cruzat, P. Newsholme, J. Cheng, Y. Chen, Y. Lu, Regulation of SIRT1 in aging: Roles in mitochondrial function and biogenesis, Mechanisms of ageing and development 155 (2016) 10-21.

1. A method of culturing and/or expanding cell or cell line or a method of preventing/inhibiting/reducing cell culture induced senescence of a cell or cell line comprising placing cell or cell line in a media comprising a nicotinamide adenine dinucleotide (NAD+) precursor; or method of rescuing a cell or cell line from senescence during culture and expansion comprising adding a NAD+ precursor to the culture media.

2. (canceled)

3. (canceled)

4. The method of claim 1, wherein the NAD precursor comprises nicotinamide (NAM), nicotinamide mononucleotide (NMN), or nicotinamide riboside (NR).

5. The method of claim 1, wherein the cell or cell line comprises stem cells

6. The method of claim 5, wherein the cell or cell line comprises human mesenchymal stem cells (hMSC), bone marrow mesenchymal stem cells (BM-MSCs), adipose derived stem cells (ASCs), umbilical cord derived stem cells (UC-MSCs); human progenitor cell line including but not limited to human endothelial progenitor cells, neuronal progenitor cells and human fibroblasts.

7. The method of claim 1, wherein the media comprises a Xeno-free culture medium.

8. The method of claim 1, wherein the NAD precursor is added to the culture after passage (P) 1 (P1), P2, P3, P4, P5, P6, P7, P8, P9, P10, P11, P12, P13, P14, P15, P16, P17, P18, P19, P20, P21, P22, P23, P24, P25, P26, P27, P28, P29, P30, P35, P40, P45, P50, P55, P60, P65, P70, P75, P80, P85, P90, P95, or P100.

9. The method of claim 1, wherein the NAD precursor is added to the culture at least one time every 12, 24, 36, 48, 60, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 60 days.

10. The method of claim 9, wherein the NAD precursor is added to the culture at least 1, 2, 3, 4, 5, 6, 7, 8, 9, 10, 11, 12, 13, or 14 times per week.

11. The method of claim 1, wherein the NAD precursor is added to the culture at least 1, 2, 3, 4, 5, 6, 7, 8, 9, 10, 11, 12, 13, or 14 times per passage.

12. The method of claim 1, wherein the cells are passaged at least once every 12, 18, 24, 30, 36, 42, 48, 54, 60, 66, 72, 78, 84, 90, 96, 102, 108, 114, 120, 126, 132, 138, 144, 150, 156, 162, or 168 hours.

13. The method of claim 1, wherein glycolysis is enhanced.

14. A culture media comprising one or more NAD precursors.

15. The culture media of claim 14, wherein the NAD precursor comprises nicotinamide (NAM), nicotinamide mononucleotide (NMN), or nicotinamide riboside (NR).

16. The culture media of claim 14, wherein the media comprises a Xeno-free culture medium.

17. The culture media of claim 14, wherein the media comprises Roswell Park Memorial Institute (RPMI) 1640, complete culture medium (CCM), Minimal Essential Medium (MEM), Dulbecco's Modified Eagle Medium (DMEM), Iscove's Modified Dulbecco's Medium (IMDM), Eagle's minimal essential medium (EMEM), Cell Therapy systems (CTS) essential 8 medium, Medium 199, essential 8 medium, StemFlex medium, and AdvanceSTEM cell culture media.
